(19) United States
(12) Patent Application Publication
(10) Pub. No.: US 2026/0248392 A1
(43) Pub. Date: Aug. 27, 2026

(54) INTERNALLY RETRACTABLE OPTICAL PROBE

(71) Applicant: Groton Consulting LLC, Derry, NH (US)

(72) Inventor: Christopher Petroff, Derry, NH (US)

(21) Appl. No.: 19/462,265

(22) Filed: Jan. 28, 2026

Related U.S. Application Data
(60) Provisional application No. 63/762,185, filed on Feb. 24, 2025.

(51) Int. Cl. A61B 5/00

(52) U.S. Cl. CPC A61B 5/0084; A61B 5/0066; A61B 5/742; A61B 2560/0443; A61B 2576/00

(57) ABSTRACT

Disclosed are imaging systems comprising imaging probes (e.g. optical imaging probes) and one or more delivery devices, such as delivery catheters and/or guidewires. The imaging probe can be configured to be positioned proximate a patient site and to collect image data from the patient site, such as a neural site, cardiac site, and/or other patient site. The imaging probe comprises an elongate shaft including a lumen. In some embodiments, a rotatable optical core and a distally positioned optical assembly are positioned within the lumen of the probe shaft. The concepts disclosed further include methods of introducing the imaging probe to a patient site, such as a neural site, using one or more delivery devices such as delivery catheters and/or guidewires. In some embodiments, the imaging probe is advanced through a delivery catheter to a patient site, without being advanced over a guidewire. Finally, the present disclosure further includes methods of measuring the effect of removing a blockage from an artery.

### CROSS-REFERENCE TO RELATED APPLICATION

This application claims priority to and the benefit of U.S. Application Ser. No. 63/762,185, filed on Feb. 24, 2025, the content of which is incorporated by reference herein in its entirety.

### BACKGROUND

### 1. Field of the Discovery

Embodiments of the disclosure presented herein relate generally to the field of imaging systems, and in particular, optical imaging systems configured for optical coherence tomography, including imaging probes.

### 2. Background Information

Optical coherence tomography (OCT) is a light-based method for imaging the topological and internal microstructure of samples in three dimensions. OCT an established medical imaging technique used across multiple medical disciplines including ophthalmology, cardiology, neurology, gastroenterology, oncology, dermatology and dentistry. OCT can be configured as a conventional microscope, as an ophthalmic scanner, or using endoscopes and small diameter catheters for accessing internal biological organs.

Regardless of the specific variety, the fundamental subsystems for all OCT instruments include a light source, an interferometer comprising reference and sample paths, a beam scanning mechanism to control the illumination of a sample, and an optical receiver coupled with signal and image processing. In simplest form, an OCT system repeatedly measures the reflectance profile of a sample along the optical axis as the illuminating beam is transversely swept across the sample. Signal processing is used to compute reflectance and ranging data that is subsequently processed to produce cross-sectional or volumetric images. OCT is unrivaled in imaging the transparent tissues of the anterior eye and retina and its compatibility with optical fiber delivery makes it uniquely suitable for imaging internal organs through minimally invasive, narrow diameter catheters and endoscopes. Thus, internal organs can be accessed by narrow diameter, flexible catheters and endoscopes that may be inserted through luminal channels or minimally invasive incisions. Since OCT catheters and endoscopes may be fabricated using flexible, narrow diameter optical fiber, it has become routine to make biocompatible, disposable probes for human internal organ imaging.

Endoscopic OCT systems generally consist of a small flexible catheter containing the necessary optical components at the tip. The catheter is attached at the end of the endoscope alongside the standard cameras. The basic function of an OCT endoscope is to deliver and focus an imaging beam on to a sample, scan the beam, collect the reflected light from the sample, and transmit it back to the OCT interferometer. Based on the direction of the imaging beam with respect to the longitudinal axis of the probe, OCT endoscopes can be divided into side-viewing endoscopes and forward-viewing endoscopes. A side-viewing endoscope is generally more suited for surveying a large area of a luminal organ, while a forward-viewing endoscope is generally more suited for image guidance of biopsies, device placement, or treatments in which a sufficient space between the OCT probe and the sample surface is needed. The distal-end optics is often housed in a metal guard, and the entire fiber may be encased in a torque coil that offers protection and flexibility. It can also transfer torque (for probe rotation) and allows for linear translation (for probe pullback) from the proximal end to the distal end. For practical use, the entire OCT endoscope is further encased in a transparent plastic sheath, which protects the probe from direct contact with body fluids and can be conveniently disinfected for human use. Based on the location of the beam scanning device, OCT endoscopes can be divided into proximal-end scanning probes and distal-end scanning probes.

Conventional OCT probes have the disadvantage that they need to be retracted to make an image along a vessel. Once the probe is retracted, access to the imaged anatomy is lost. It is difficult to readvance to get to the imaged anatomy. Thus, there is a need for imaging systems that can create an image without losing distal access, as well as systems with one or more delivery devices compatible with these improved imaging probes.

### SUMMARY

Disclosed are imaging systems comprising imaging probes and one or more delivery devices, such as delivery catheters and/or guidewires. The imaging probe can be configured to be positioned proximate a patient site and to collect image data from the patient site, such as a neural site, cardiac or circulatory system site, spinal site and/or other patient site as defined or exemplified herein. The imaging probe comprises an elongate shaft including a lumen. In some embodiments, a rotatable optical core is distally positioned on an optical assembly within the lumen of the probe shaft and can translate in the probe shaft. The disclosure further includes methods of introducing the imaging probe to a patient site, such as a neural site, using one or more delivery devices such as delivery catheters and/or guidewires. The present disclosure further includes methods of determining if an imaged flow blockage is significant.

In one embodiment, the disclosure includes an imaging system comprising a first delivery device with a first elongate shaft having a first proximal end, a first distal end, and a first lumen extending between the first proximal end and the first distal end; an imaging probe with a second elongate shaft having a second proximal end, a distal portion, and a second lumen extending between the second proximal end and the distal portion; a rotatable optical component positioned within the second lumen of the second elongate shaft and having a third proximal end and a second distal end; and an optical assembly positioned within the distal portion of the second elongate shaft and near the second distal end of the rotatable optical component, wherein the optical assembly is configured to direct light to tissue and collect reflected light from the tissue. The system further includes an interface unit configured to optically and mechanically connect to the rotatable optical component, wherein the interface unit comprises a rotating assembly constructed and arranged to rotate the optical assembly and a retraction assembly constructed and arranged to retract the optical assembly separate from the second elongate shaft. The first elongate shaft and the second elongate shaft are configured to translate separately.

In another embodiment, the system includes the translation of the first elongate shaft and the second elongate shaft comprising simultaneous insertion of the first elongate shaft and the second elongate shaft into a patient. The system may further comprise a second delivery device with a third elongate shaft having a fourth proximal end, a third distal end, and a third lumen between the fourth proximal end and the third distal end, wherein the first elongate shaft and the second elongate shaft are constructed and arranged to translate within the third lumen. The first elongate shaft may comprise a first outer diameter and the third elongate shaft may comprise a first inner diameter, wherein the first inner diameter is larger than the first outer diameter.

In a further embodiment, the first delivery device and the imaging probe are configured to frictionally engage, wherein the frictional engagement is configured to maintain relative position between the first elongate shaft and the second elongate shaft. The first lumen of the first delivery device may comprise a closed distal end, and the first elongate shaft may further comprise a transparent segment.

The present disclosure provides an advanced imaging system designed for medical applications, particularly for intravascular and neurological imaging. The system includes a first delivery device having an elongate shaft with a proximal end, a distal end, and a lumen extending between these ends. This delivery device is configured to receive and guide an imaging probe to a target site within a patient.

The imaging probe itself comprises a second elongate shaft with its own proximal end, a distal portion, and a second lumen extending from the proximal end to the distal portion. Within this second lumen is positioned a rotatable optical core, which has a proximal end and a distal end. At the distal portion of the second elongate shaft, and proximate the distal end of the rotatable optical core, is an optical assembly. This optical assembly is configured to direct light to tissue and collect reflected light, enabling high-resolution imaging of internal structures.

The system further includes an interface unit that optically and mechanically connects to the rotatable optical core. The interface unit comprises a rotating assembly, which rotates the optical assembly, and a retraction assembly, which is constructed to retract the optical assembly independently of the second elongate shaft. The first and second elongate shafts are configured to translate separately, allowing for flexible and precise positioning within the patient.

In certain embodiments, the system allows for simultaneous insertion of both the first and second elongate shafts into a patient. The system may also include a second delivery device with a third elongate shaft, having its own proximal and distal ends and a lumen extending between them. The first and second elongate shafts can be constructed and arranged to translate within this third lumen, with the third elongate shaft having an inner diameter larger than the outer diameter of the first elongate shaft.

Additional embodiments provide for frictional engagement between the first delivery device and the imaging probe, maintaining their relative positions during use. The first lumen of the delivery device may have a closed distal end, and the first elongate shaft may include a transparent segment, which can range in length from 1 cm to 20 cm, to facilitate optical imaging.

The retraction assembly can retract the optical assembly at rates between 5 mm/sec and 150 mm/sec, with a preferred rate of approximately 75 mm/sec. The pullback procedure performed by the retraction assembly may involve retracting only the optical assembly, not the second elongate shaft, over 20 mm to 150 mm and within a time of 1 to 15 seconds.

The system is designed with flexibility in mind, allowing for components that are either disposable or reusable, with reusable components being sterilizable. The system may further include a display configured to provide one or more images based on the light collected by the optical assembly, supporting real-time visualization and analysis during medical procedures.

In certain embodiments, the system further comprises a processor operatively coupled to the imaging probe. The processor is configured to receive image data collected by the optical assembly from a patient site before and after a treatment procedure, determine a flow recovery ratio by comparing flow measurements derived from the image data acquired before and after the treatment, and output the flow recovery ratio to a display.

A method of operating the system is also provided. The method includes acquiring first image data from a patient site using the optical assembly prior to a treatment procedure, acquiring second image data from the patient site after the treatment procedure, calculating a flow recovery ratio by comparing flow measurements derived from the first and second image data, and displaying the flow recovery ratio to a user. In some embodiments, the calculation of the flow recovery ratio (FRR) is performed as the ratio of the flow measurement before the treatment procedure (Fₚᵣₑ) to the flow measurement after the treatment procedure (Fₚₒₛₜ), such that:

$$FRR = \frac{{F}_{p r e}}{{F}_{post}}$$

where Fₚᵣₑ and Fₚₒₛₜ are determined from velocity and cross-sectional area measurements derived from the first and second image data, respectively. This enables clinicians to quantitatively assess the effectiveness of a treatment procedure in restoring flow at the patient site.

The preceding general areas of utility are given by way of example only and are not intended to be limiting on the scope of the present disclosure and appended claims. Additional objects and advantages associated with the compositions, methods, and processes of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference.

### BRIEF DESCRIPTION OF THE DRAWINGS

The accompanying drawings, which are incorporated into and form a part of the specification, illustrate several embodiments of the present disclosure; and, together with the description, explain the principles of the disclosure. The drawings are only for the purpose of illustrating embodiments of the disclosure and are not to be construed as limiting the disclosure. The drawings are not necessarily to scale; emphasis is placed instead upon illustrating the principles of disclosed embodiments. Further objects, features and advantages of the disclosure will become apparent from the following detailed description taken in conjunction with the accompanying figures showing illustrative embodiments of the disclosure, in which:

FIG. 1 is a schematic view of an imaging system comprising an imaging probe, console and one or more delivery devices, consistent with the present disclosed concepts.

FIG. 2 is a sectional view of an imaging probe wherein the imaging probe comprises an optical assembly configured to collect image data while positioned outside the delivery catheter.

FIG. 3 is a sectional view of delivery catheters consistent with the present disclosed concepts.

FIG. 4 is a view of guidewires consistent with the present disclosure.

FIG. 5 is a flow chart of a method for creating an image, including advancing an imaging probe beyond the distal end of a delivery catheter prior to collecting image data and readvancing the first delivery catheter over the imaging probe, consistent with the present disclosure steps.

FIG. 6 Shows the arteries of the brain.

FIG. 7 is a schematic anatomical view of Step 3 504 of the method of FIG. 5

FIG. 8 is an illustration of Step 4 506 of the method of FIG. 5

FIG. 9 is an illustration of Step 5 508 of the method of FIG. 5

FIG. 10 is an illustration of Step 6 510 of the method of FIG. 5

FIG. 11 is an illustration of Step 7 512 of the method of FIG. 5

FIG. 12 is an illustration of Step 8 514 of the method of FIG. 5

FIG. 13 is an illustration of Step 10 518 of the method of FIG. 5

FIG. 14 is a flow chart of a method for when the delivery device remains proximal to the patient site.

FIG. 15 is an illustration of Step 3 1404 of the method of FIG. 14

FIG. 16 is an illustration of Step 4 1406 of the method of FIG. 14

FIG. 17 is an illustration of Step 5 1408 of the method of FIG. 14

FIG. 18 is an illustration of Step 6 1410 of the method of FIG. 12

FIG. 19 is a flow chart of a method to create an image with the smallest catheter removed.

FIG. 20 is an illustration of Step 3 1904 of the method of FIG. 19

FIG. 21 is an illustration of Step 4 1906 of the method of FIG. 19

FIG. 22 is an illustration of Step 5 1908 of the method of FIG. 19

FIG. 23 is an illustration of Step 6 1910 of the method of FIG. 19

FIG. 24 is an illustration of Step 7 1912 of the method of FIG. 19

FIG. 25 is a flow chart of a method to image during treatment of the patient site.

FIG. 26 is an illustration of Step 7 2512 of the method of FIG. 25

FIG. 27 is a sectional view of a delivery device with side holes.

FIG. 28 is a sectional view of flushing fluid from delivery catheter C.

FIG. 29, a schematic view of a probe comprising, a rotating optical core 2900 is illustrated with the rotating optical core 2900 in its most distal position, consistent with the present disclosed concepts.

FIG. 30 is a sectional view of the imaging probe with the rotating optical core retracted.

FIG. 31 is a schematic view of a system comprising an imaging probe, a rotating assembly and a retraction and advancing assembly with the assembly advanced, consistent with the present disclosure.

FIG. 32 is a schematic view of a system comprising an imaging probe, a rotating assembly and a retraction and advancing assembly with the assembly retracted, consistent with the present disclosure.

FIG. 33 is a sectional view of the imaging probe.

FIG. 34 is a sectional view of semi-solid and liquid in the imaging catheter.

FIG. 35 is the rheologic curve of semi solid.

FIG. 36 is a sectional view of semi-solid and liquid when semi solid is injected a probe shaft joint.

FIG. 37 is an illustration of turbine design to reduce fiber stress when rotating optical core travels.

FIG. 38 is an illustration of the fiber mover located when the optical core is advanced.

FIG. 39 is an illustration of the fiber mover located when the optical core is retracted.

FIG. 40 is a sectional view showing the optical assembly diameter relative to the rotating image core diameter.

FIG. 41 is a sectional view of the optical assembly with an angled reflector on the graded index fiber.

FIG. 42 is a sectional view of the optical assembly with an angled reflector on the coreless fiber.

FIG. 43 is an illustration of a curved polish on the graded index fiber.

FIG. 44 is an illustration of the irradiance from the curved polish at the beam waist.

FIG. 45 is an illustration of a flexible optical assembly.

FIG. 46 is an illustration of a flexible optical assembly with a coreless section.

FIG. 47 is an illustration of a variable stiffness transparent sheath section.

FIG. 48 is an illustration of an assembly step of a variable stiffness transparent sheath section.

FIG. 49 is an illustration of a variable stiffness window made from a single extrusion.

FIG. 50 is an illustration of a variable stiffness window made from a tapered extrusion.

FIG. 51 is an illustration of a variable stiffness window made from an embedded coil.

FIG. 52 is an illustration the proximal window joint made from a tube on the inside of the probe shaft.

FIG. 53 is an illustration of the tube cover bent over a metal tube.

FIG. 54 is an illustration of the tube buffer inside the tube cover in a bent probe shaft.

FIG. 55 is an illustration the proximal window joint made from the tube cover.

FIG. 56 is an illustration of a polymer tip attached to the transparent section without a core wire.

FIG. 57 is an illustration of a polymer tip attached to the transparent section using a core wire.

FIG. 58 is an illustration of a J shaped polymer tip.

FIG. 59 is an illustration of a U-shaped polymer tip.

FIG. 60 is an illustration of the vessel division for pressure recovery calculation after treatment.

FIG. 61 is a flow chart of the method to calculate pressure recovery.

FIG. 62 is an illustration of the calculation method for pressure recovery.

FIG. 63 is an illustration of the flow recovery measurement locations.

FIG. 64 is a flow chart of the method to calculate flow recovery.

### DETAILED DESCRIPTION

An imaging system is provided. The system includes a first delivery device having a first elongate shaft with a first proximal end, a first distal end, and a first lumen extending between the proximal and distal ends. The system further includes an imaging probe. The imaging probe comprises a second elongate shaft with a second proximal end, a distal portion, and a second lumen extending between the second proximal end and the distal portion. A rotatable optical core is positioned within the second lumen of the second elongate shaft and has a third proximal end and a second distal end. An optical assembly is positioned within the distal portion of the second elongate shaft and proximate the second distal end of the rotatable optical core. The optical assembly is configured to direct light to tissue and collect reflected light from the tissue. The system also includes an interface unit configured to optically and mechanically connect to the rotatable optical core. The interface unit comprises a rotating assembly constructed and arranged to rotate the optical assembly, and a retraction assembly constructed and arranged to retract the optical assembly separate from the second elongate shaft. The first elongate shaft and the second elongate shaft are configured to translate separately.

While various embodiments of the present disclosure are described herein, it will be understood by those skilled in the art that such embodiments are provided by way of example only. It will be understood by those skilled in the art that numerous modifications and changes to, and variations and equivalent substitutions of, the embodiments described herein can be made without departing from the scope of the disclosure. It is understood that various alternatives to the embodiments described herein may be employed in practicing the disclosure, and modifications may be made to adapt a particular structure or material to the teachings of the disclosure. It is also understood that every embodiment of the disclosure may optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

Unless otherwise defined, all technical and scientific terms used herein have the same meaning as commonly understood by one of ordinary skill in the art to which this disclosure belongs.

Where a range of values is provided, it is understood that each intervening value between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both limits, ranges excluding both of those included limits are also included in the disclosure.

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The term “exemplary” as used herein means “serving as an example, instance or illustration”. Any embodiment or feature characterized herein as “exemplary” is not necessarily to be construed as preferred or advantageous over other embodiments or features.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either” “one of,” “only one of,” or “exactly one of.”

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, in certain methods described herein that include more than one step or act, the order of the steps or acts of the method is not necessarily limited to the order in which the steps or acts of the method are recited unless the context indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

As described herein, “room pressure” shall mean pressure of the environment surrounding the systems and devices of the present disclosed concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component where used herein are the length and diameter, respectively, of the smallest volume hypothetical cylinder which can surround the component.

The term “transducer” where used herein is to be taken to include any component or combination of components that receives energy or any input and produces an output. For example, a transducer can include an electrode that receives electrical energy and distributes the electrical energy to tissue (e.g. based on the size of the electrode). In some configurations, a transducer converts an electrical signal into any output, such as light (e.g. a transducer comprising a light emitting diode or light bulb), sound (e.g. a transducer comprising a piezo crystal configured to deliver ultrasound energy), pressure, heat energy, cryogenic energy, chemical energy; mechanical energy (e.g. a transducer comprising a motor or a solenoid), magnetic energy, and/or a different electrical signal (e.g. a Bluetooth or other wireless communication element). Alternatively, or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “patient site” refers to a location within the patient, such as a location within a body conduit such as a blood vessel (e.g. an artery or vein such as an artery or vein of the heart) or a segment of the GI tract (e.g. the esophagus, stomach or intestine), or a location within an organ. A “patient site” can refer to a location in the spine, such as within the epidural space or intrathecal space of the spine. A patient site can include a location including one or more of: an aneurysm; a stenosis; thrombus and/or an implant.

As used herein, the term “neural site” refers to a patient site proximate the brain, such as at a location within the neck, head or brain of a patient. A neural site can include a location proximate the brain including one or more of: an aneurysm; a stenosis; thrombus and/or an implant.

As used herein, the term “proximate” shall include locations relatively close to, on, in and/or within a referenced component or other location. In similar fashion, the term “distal” shall include locations relatively distant from, on, in, and/or within a referenced component or other location.

As used herein, the term “transparent” and “optically transparent” refer to a property of a material that is relatively transparent (e.g. not opaque) to light delivered and/or collected by one or more components of the imaging system or probe of the present disclosed concepts (e.g. to collect image data). The term “translucent” may also account for such a property.

As used herein, the term “algorithm,” refers not only to the traditionally used sense, but also, in certain embodiments to any required computer elements (e.g., RAM, ROM, tangible non-transitory computer memory, processors, and input/output devices) required to execute the algorithm in the context of embodiments consistent with the disclosure.

### Selected Abbreviations:

OCT optical coherence tomography
PRR pressure recovery ratio
FRR flow recovery ratio
rps revolutions per second
Fr French—catheter unit of diameter measurement

This disclosure introduces imaging systems comprising imaging probes (e.g. optical imaging probes) and one or more delivery devices, such as delivery catheters and/or guidewires. The imaging probe can be configured to be positioned proximate a patient site and to collect image data from the patient site, such as: a neural site, a cardiac or circulatory system site, and/or other patient site. The imaging probe comprises an elongate shaft including a lumen. In some embodiments, a rotatable optical core and a distally positioned optical assembly are positioned within the lumen of the probe shaft. The present disclosed concepts further include methods of introducing the imaging probe to a patient site, such as a neural site, using one or more delivery devices such as delivery catheters and/or guidewires. In some embodiments, the imaging probe is advanced through a delivery catheter to a patient site, without being advanced over a guidewire. Finally, the present disclosure further includes methods of measuring the effect of removing a blockage from an artery.

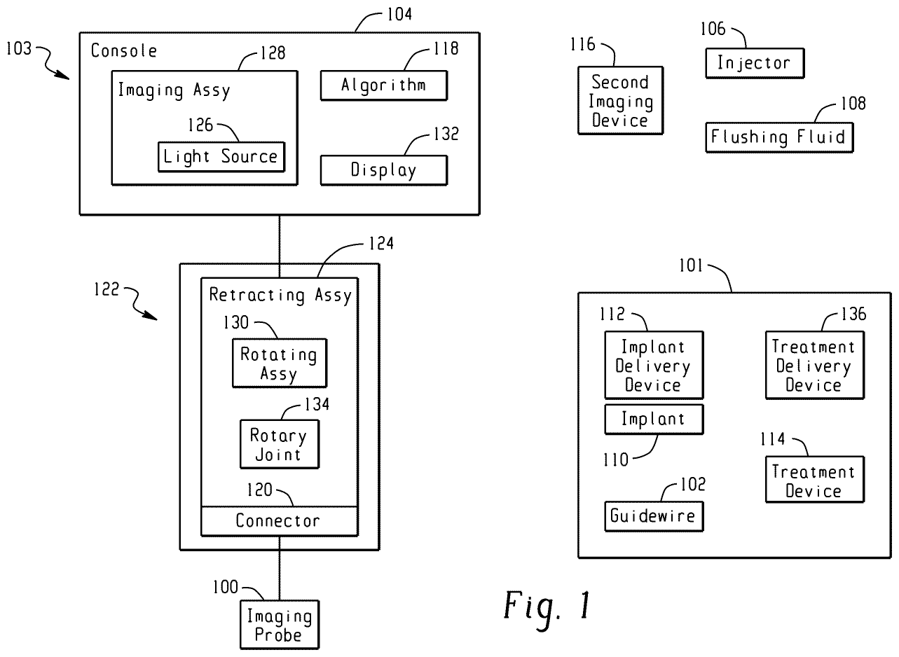

FIG. 1 presents a block diagram schematic view of an imaging system comprising an imaging probe and one or more delivery devices. “System” comprises everything in the figure. System is constructed and arranged to collect image data and produce an image based on the recorded data, such as when system comprises an Optical Coherence Tomography (OCT) imaging system. System comprises “imaging probe” 100, and at least one delivery device, such as at least one delivery catheter 101 and/or at least one guidewire 102. System 103 can further comprise console 104 which is configured to operably attach to imaging probe 100 through a bedside unit 122. The console 104 and bedside unit 122 may be integrated together or separate components mechanically and/or electrically or otherwise operably connected. System 103 can further comprise a fluid injector, such as injector 106 which can be configured to inject one or more fluids, such as a flushing fluid, an imaging contrast agent (e.g. a radiopaque contrast agent, hereinafter “contrast”) and/or other fluid, such as flushing fluid 108 shown schematically. System 103 can further comprise an implant, such as implant 110 which can be implanted in the patient via treatment diagnostic/delivery device 136. System 103 can further comprise a device configured to treat the patient, treatment device 114, which can be configured to dilate a stenotic site, remove stenotic material (e.g. thrombus) and/or otherwise treat a patient disease or disorder. System 103 can further comprise a second imaging device, such as imaging device 116 shown as a schematic representation.

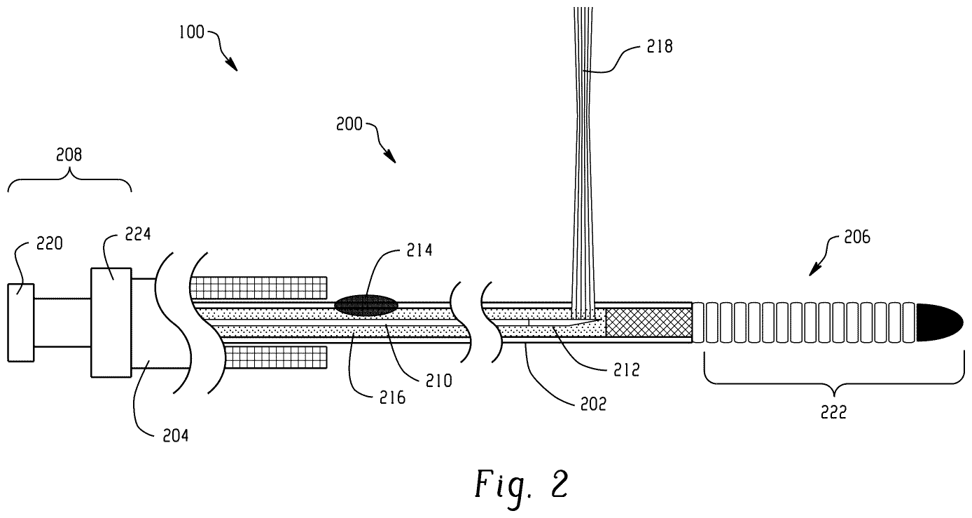

Referring now to FIG. 2, imaging probe 100 comprises an elongate shaft, shaft 202, comprising a proximal end 204, a distal end 206, a mid-portion (located about elements 210, 214, and 216), and a distal portion (located to the right of about element 218). It should be noted that the terms “proximal,” “mid,” and “distal,” connote a relative positional arrangement and do not dictate the inclusion or exclusion of any element or the creation of any particular grouping or sub-grouping. A connector 208 is positioned on the proximal end 204 of shaft 202 and is configured to operably attach probe 200 to console 104. Imaging probe 100 is configured to provide a patient image (e.g. a three-dimensional image created when rotating optical core 210 is retracted in shaft 202 of imaging probe 100). Imaging probe 100 is constructed and arranged to collect image data from a patient site. In these embodiments, the distal portion 206 can be configured to pass through the patient site, such as a patient site including occlusive material such as thrombus or a patient site including an implant.

In some embodiments, probe 200 is constructed and arranged to collect image data from a neural site, such as a neural site selected from the group consisting of: artery of patient's neck; vein of patient's neck; artery of patient's head; vein of patient's head; artery of patient's brain; vein of patient's brain; and combinations of one or more of these. In some embodiments, probe 200 is constructed and arranged to collect image data from one or more locations along or otherwise proximate the patient's spine. In some embodiments, probe 200 is constructed and arranged to collect image data from tissue selected from the group consisting of: wall tissue of a blood vessel of the patient site; thrombus proximate the patient site; occlusive matter proximate the patient site; a blood vessel outside of blood vessel in which optical assembly 212 is positioned; tissue outside of blood vessel in which optical assembly 212 is positioned; extracellular deposits outside of the lumen of the blood vessel in which optical assembly 212 is positioned (e.g. within and/or outside of the blood vessel wall); and combinations of one or more of these.

Alternatively, or additionally, optical assembly 212 can be constructed and arranged to collect image data from an implanted device (e.g. a temporary or chronically implanted device), such as an implant described herein or a device previously implanted in the patient. In some embodiments, optical assembly 212 is constructed and arranged to collect image data regarding a placement procedure in which an implant was positioned within a patient (e.g. real time data collected during placement).

Optical assembly 212, shown emitting light 218, can be constructed and arranged to collect implant data comprising position and/or expansion data related to placement of an implant or other treatment device, such as a device selected from the group consisting of: a stent retriever (also known as a stentriever); an embolization device such as an embolization coil; an occlusion device; a flow diverter; and combinations of one or more of these. In some embodiments, optical assembly 212 is constructed and arranged to collect data related to the position of an implant 110 or other device comprising a stimulation element, such as an electrode or other stimulation element positioned proximate the brain (e.g. an electrode positioned in the deep brain or other brain location) or a stimulation element positioned proximate the spine (e.g. stimulation element configured to treat pain by stimulating spine tissue).

Implantation of implant 110 can be performed based on an analysis of collected image data (e.g. an analysis of collected image data by algorithm 118). The analysis can be used to modify an implantation parameter selected from the group consisting of: selection of the implantable device (e.g. selection of implant); selection of the implantable device porosity; selection of the implantable device coverage (e.g. percentage of the surface area of vessel covered by metal or other material of the implantable device); selection of the implantable device pore density; selection of the implantable device diameter; selection of the implantable device length; selection of the location to implant the implantable device; a dilation parameter for expanding the implantable device once implanted; a repositioning of the implantable device once implanted; selection of a second implantable device to be implanted; amount the occlusion is blocking flow and combinations thereof. An adjustment of the implantation can be performed based on one or more issues identified in the analysis, such as an issue selected from the group consisting of: malposition of implanted device; inadequate deployment of implanted device; presence of air bubbles; and combinations thereof. In some embodiments, optical assembly 212 is constructed and arranged to collect data related to the position of a treatment device, such as treatment device 91 described herein, during a patient treatment procedure.

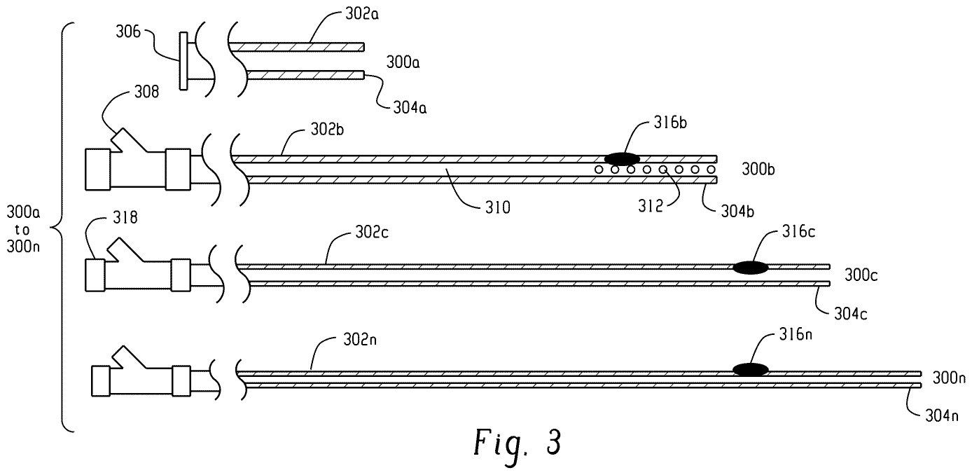

Shown in FIG. 3, delivery catheters 300 can comprise one or more delivery catheters, such as delivery catheters 300a, 300b, 300c through 300n. As shown in this figure, and used in subsequent figures and nomenclature, parts and elements with “a,” “b,” “c,” “n” style designations indicate that one or more versions exist thus, “n” may represent a putative element 300d, 300e, 300f, . . . 300z . . . etc. . . . . Thus, a reference to an “nth” type embodiment encompasses, if necessary, all of the intervening elements in the set and may also implicate an “n+1” type embodiment. In certain other embodiments the “n” designation may refer to the last, or terminal, element in the series or sequence. For example, a terminal delivery catheter may be element 300n as it is the last one in a sequence of delivery catheters.

Delivery catheters 300a-n can include a vascular introducer component. By way of example, delivery catheter 300a comprises a vascular introducer 306 (partially illustrated). Other delivery catheters 300b-n can be inserted into the patient through delivery catheter 300a, after the vascular introducer 306 is positioned through the skin of the patient. Two or more delivery catheters 300 can collectively comprise sets of inner diameters (IDs) and outer diameters (ODs) such that a first delivery catheter 300a slidingly receives a second delivery catheter 300b (e.g. the second delivery catheter OD is less than or equal to the first delivery catheter ID), and the second delivery catheter 300b slidingly receives a third delivery catheter 300c (e.g. the third delivery catheter OD is less than or equal to the second delivery catheter ID), and so on. In these configurations, the first delivery catheter 300a can be advanced to a first anatomical location, the second delivery catheter 300b can be advanced through the first delivery catheter to a second anatomical location distal or otherwise remote to the first anatomical location, and so on as appropriate, using sequentially smaller diameter delivery catheters 300n.

Each delivery catheter 300 comprises a shaft 302 (e.g. shafts 302a, 302b, 302c and 302n shown), each with a distal end 304 (e.g. distal ends 304 a, 304 b, 304 c and 304 n shown). A connector 318 is positioned on the proximal end of each shaft 302. Each connector 318 can comprise a Touhy or other valved connector, such as a valved connector configured to prevent fluid egress from the associated delivery catheter 300 (with and/or without a separate shaft positioned within the connector 318). Each connector 318 can comprise a port 308, such as a port constructed and arranged to allow introduction of fluid into the associated delivery catheter 300 and/or for removing fluids from an associated delivery catheter 300. In some embodiments, a flushing fluid, as described herein, is introduced via one or more ports 308, such as to remove blood or other undesired material from locations proximate optical assembly 212. Port 308 can be positioned on a side of connector 318 and can include a luer fitting and a cap and/or valve. Shafts 302, connectors 318 and ports 308 can each comprise standard materials and be of similar construction to commercially available introducers, guide catheters, diagnostic catheters, intermediate catheters and microcatheters used in interventional procedures.

Each delivery catheter 300 comprises a lumen 310 (shown on delivery catheter 300b but removed from the remaining delivery catheters 300 for illustrative clarity) extending from the connector 318 to the distal end 304b of shaft 302b. The diameter of each lumen 310 defines the ID of the associated delivery catheter 300. Each delivery catheter 300 can be advanced over a guidewire (e.g. guidewire 102) via lumen 310. In some embodiments, imaging probe 100 and at least one delivery catheter 300 are cooperatively constructed and arranged such that the delivery catheter 300 is advanced through a vessel, such as a blood vessel, and probe 200 is slidingly received by the delivery catheter 300 and advanced through the lumen 310 of delivery catheter 300 to a location proximate a patient site to be imaged (e.g. a location just distal to, within and/or just proximate the patient site to be imaged). In some embodiments, a second delivery catheter 300 b is slidingly received by a first delivery catheter 300 a, and probe 200 is advanced through the second delivery catheter 300 b to a location proximate a patient site to be imaged. In yet other embodiments, three or more delivery catheters 300 are coaxially inserted into each other, with probe 200 advanced through the innermost delivery catheter 300 to a location proximate a patient site to be imaged. In some embodiments, probe 200 is advanced through (e.g. through and beyond) one or more delivery catheters 300 without the use of a guidewire.

Delivery catheters 300 can comprise one or more delivery catheters selected from the group consisting of: an introducer; a vascular introducer (partially illustrated as element 306); an introducer with an ID between 7 Fr and 9 Fr; a delivery catheter (also referred to as a guide catheter) for positioning through the aortic arch (e.g. such that it's distal end is just distal or otherwise proximate the aortic arch) such as a delivery catheter with an ID between 5 Fr and 7 Fr or an ID of approximately 6.5 Fr; a delivery catheter (also referred to as an intermediate catheter) for insertion through a larger, previously placed delivery catheter, such as an intermediate delivery catheter with an ID of between 0.053″ and 0.070″; a delivery catheter (also referred to as a microcatheter) with an ID of between 0.0165″ and 0.027″; and combinations of one or more of these. In some embodiments, delivery catheters 300 comprise a first delivery catheter 300a comprising an introducer 306, such as an introducer with an ID of between 7 Fr and 9 Fr or an ID of approximately 8 Fr. Delivery catheters 300 can further comprise a second delivery catheter 300b constructed and arranged to be inserted into the first delivery catheter 300a. Second delivery catheter 300b, can be constructed and arranged for positioning through the aortic arch and can comprise an ID between 5 Fr and 7 Fr or an ID of approximately 6.5 Fr. Delivery catheters 300 can comprise a third delivery catheter 300c constructed and arranged to be inserted through the first delivery catheter 300a and/or the second delivery catheter 300b. A third delivery catheter 300c may comprise an ID of between 0.053″ and 0.070″. Delivery catheters 300 can comprise a fourth delivery catheter 300n constructed and arranged to be inserted through the first, second and/or third delivery catheters 300a-c. The fourth delivery catheter 300n may comprise an ID of between 0.0165″ to 0.027″ This sequence may continue for any n amount of delivery catheters as required and/or physically practical.

Imaging probe 100 can be constructed and arranged to be inserted through first, second, third and/or fourth (or “n” more) delivery catheters 300, such as when imaging probe 100 comprises an OD of less than 0.070″, such as when at least the distal portion of imaging probe 100 comprises an OD of less than or equal to 0.025″, 0.022″, 0.018″, 0.016″, 0.015″ or 0.014″. In some embodiments, at least the distal portion of imaging probe 100 comprises an OD of approximately 0.014″ (e.g. an OD between 0.013″ and 0.017″). In some embodiments, system 103 comprises a probe 200 and one or more delivery catheters 300 as described herein in reference to FIGS. 2 and 3.

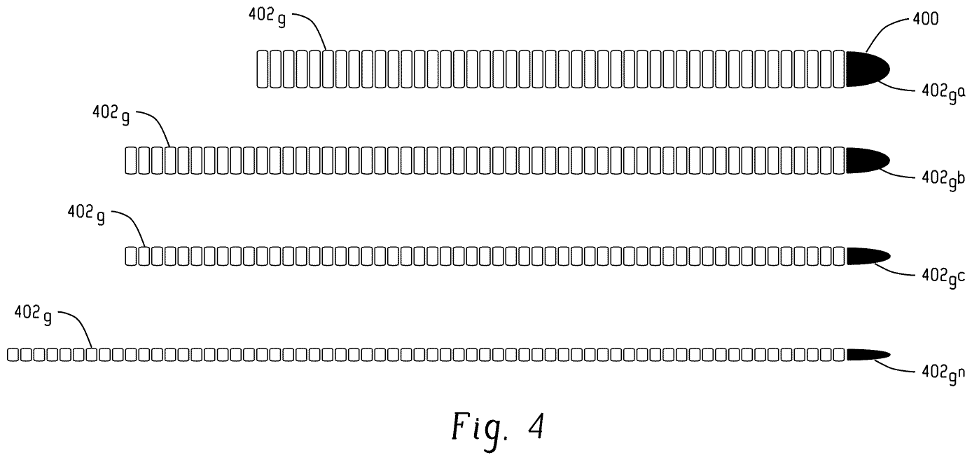

Illustrated in FIG. 4, Each delivery catheter 300 can comprise a spring tip 400. Guidewires 402g can comprise one or more guidewires, such as guidewires 402ga, 402gb, 402gc through 402g n. Guidewires 402g can comprise one or more guidewires constructed and arranged to support advancement (e.g. intravascular advancement) of imaging probe 100 (e.g. via a rapid exchange lumen in distal portion 304 of shaft 302) and/or a delivery catheter 300 into a patient site such as a neural site. Guidewires 402g can comprise one or more guidewires selected from the group consisting of: a guidewire with an OD between 0.035″ and 0.038″; a guidewire with an OD between 0.010″ and 0.018″; an access length guidewire such as a guidewire with a length of approximately 200 cm; an exchange length guidewire such as a guidewire with a length of approximately 300 cm; a guidewire with a length between 175 cm and 190 cm; a guidewire with a length between 200 cm and 300 cm and/or an OD between 0.014″ and 0.016″; a hydrophilic guidewire; a Stryker Synchro™ guidewire; a Terumo™ guidewire such as the Terumo Guidewire™ guidewire; a Terumo Traxcess™ guidewire; an X-Celerator™ guidewire; an X-Pedion™ guidewire; an Agility™ guidewire; a Bentson™ guidewire; a Coon™ guidewire; an Amplatz™ guidewire; and combinations of one or more of these. In some embodiments, system 103 comprises an imaging probe 100 and one or more guidewires 402ga-n as described herein in reference to FIGS. 2, 3 and 4. Guidewires 402g can comprise one or more visualizable portions, such as one or more radiopaque or ultrasonically reflective portions.

System 103 can comprise various sets and configurations of delivery catheters 300 and guidewires 402g. In some embodiments, delivery catheters 300 comprise a first delivery catheter 300a comprising an introducer (e.g. a vascular introducer 306), and at least two delivery catheters 300 that are inserted through delivery catheter 300, these catheters comprising corresponding different sets of IDS and ODS, such as to allow sequential insertion of each delivery catheter 300 through the lumen 310 of a previously placed delivery catheter 300, as described in detail herein. In some embodiments, a first delivery catheter 300a is advanced over a first guidewire 402ga, and a smaller OD delivery catheter 300b is subsequently advanced over a smaller OD guidewire 402gb (e.g. after the first guidewire 402ga is removed from the first delivery catheter 300 and replaced with the second guidewire 402gb).

In some embodiments after image data is collected by an imaging probe 100 positioned within a delivery catheter, the delivery catheter is readvanced over the imaging probe, imaging probe 100 is removed and replaced with a guidewire 402g over which an additional device can be placed (e.g. another delivery catheter 300, a treatment device 114, a treatment diagnostic/delivery device 136 or other device). In some embodiments, probe 200, one or more delivery catheters 300 and/or one or more guidewires 402g are inserted, advanced and/or retracted as described herein in reference to FIGS. 2, 3 and 4.

In some embodiments, one or more delivery catheters 300 comprise a functional element 316 (e.g. functional elements 316b, 316c and 316n shown). Each functional element 316 can comprise one or more functional elements such as one or more sensors, transducers and/or other functional elements as described in detail herein.

In some embodiments, one or more delivery catheters 300 comprise a transparent section (not shown). Each transparent section can comprise one or more functional elements 316 such as one or more sensors, transducers and/or other functional elements as described in detail herein.

Referring additionally to FIG. 2, a magnified view of proximal end 204 and distal end 206 is illustrated, consistent with the present disclosed concepts. A lumen 216 extends from proximal end 204 of shaft 202 to distal end 206, ending at a location proximal to distal end 206. Positioned within lumen 216 is a rotating optical core, core 210. An optical assembly 212 is positioned on the distal end of core 210. Optical assembly 212 can include light beam 218 which translates and rotates within an optically translucent and/or effectively transparent portion of shaft 202. Optical assembly 212 is constructed and arranged to collect image data through at least a portion of shaft 202 (e.g. through transparent portion). In some embodiments, optical assembly 212 is further constructed and arranged to collect image data through at least a portion of an additional device, such as at least a portion of a shaft of a delivery catheter 300 (e.g. an optically transparent portion of a delivery catheter 300). Translating section of connector 208 can be rotatably attached to optical core 210 (e.g. allowing rotation of core 210 relative to one or more portions of connector 208 and allowing longitudinal translation of core 210 relative to fixed section of connector 208). In some embodiments, fixed section of connector 208 is fixedly attached to shaft 202, such as to prevent all relative motion between connector 208 and shaft 202. In some embodiments, a separate connector that translates is included, called the connector translating section 220. Connector translating section 220 is fixedly attached to rotating optical core 210, in a manner such that the rotational and longitudinal motion of the connector translating section 220 and the rotating optical core 210 are in unison. In some embodiments, the connector translating section 220 is fixedly attached to the rotating optical core 210, so that they spin in unison. In some embodiments, the connector translating section 220 is fixedly attached to the rotating optical core, so that they spin and translate in unison. In all these embodiments the connector translating section 220 is fixedly attached to rotating optical core 210, such to allow relative motion between the rotating optical core 210 and the shaft 202.

Probe 200 is configured to collect image data, such as image data collected during rotation and/or retraction of optical assembly 212. Optical assembly 212 can be rotated by rotating optical core 210. Optical assembly 212 can be retracted by translating section of connector 208. Optical assembly 212 can collect image data while surrounded by a portion of a shaft of a delivery catheter 300 (e.g. when within a transparent segment of a delivery catheter) and/or when there is no delivery catheter 300 segment surrounding optical assembly 212 (e.g. when optical assembly 212 has been advanced beyond the distal ends 206 of all delivery catheters 300 into which probe 200 is inserted.

During collection of image data, a flushing procedure can be performed, such as by delivering one or more fluids, flushing fluid 108 (e.g. as propelled by injector 106 or other fluid delivery device), to remove blood or other somewhat opaque material (hereinafter non-transparent material) proximate optical assembly 212 (e.g. to remove non-transparent material between optical assembly 212 and a delivery catheter and/or non-transparent material between optical assembly 212 and a vessel wall), such as to allow light distributed from optical assembly 212 to reach and reflectively return from all tissue and other objects to be imaged. In these flushing embodiments, flushing fluid 108 can comprise an optically transparent material, such as saline. Flushing fluid 108 can comprise one or more visualizable materials, as described herein. Flushing fluid 108 can be delivered by injector 106 as described herein.

Alternative or in addition to its use in a flushing procedure, flushing fluid 108 can comprise material configured to be viewed by second imaging device 116, such as when flushing fluid 108 comprises a contrast material configured to be viewed by a second imaging device 116 comprising a fluoroscope or other X-ray device; an ultrasonically reflective material configured to be viewed by a second imaging device 116 comprising an ultrasound imager; and/or a magnetic material configured to be viewed by a second imaging device 116.

Flushing fluid 108 can be delivered by one or more delivery catheters 300 (e.g. in the space between a first delivery catheter 300a and an inserted/second delivery catheter 300b, or in the space between a delivery catheter 300 and an inserted probe 200 or in the space between a delivery catheter 300 and the imaging probe 100). Flushing fluid 108 delivered in a flushing procedure can be delivered out the distal end 304 of a delivery catheter 300 (e.g. a distal end 304 positioned proximal to optical assembly 212), as described herein in reference to FIGS. 9-28. Alternatively, or additionally, any delivery catheter 300 can comprise one or more sideholes 312 (illustrated schematically for one embodiment, it is understood the sideholes 312 may be one or more, spaced in different arrangements, take different shapes, etc.) passing through a portion of the associated shaft 302, such as sideholes 312 shown positioned on a distal portion of delivery catheter 300b. In some embodiments, a delivery catheter 300 comprises a delivery catheter comprising sideholes 312 positioned in a distal portion, such as a microcatheter with an ID more than 0.027″ (e.g. a delivery catheter with an ID between 0.027″ and 0.0150″). In some embodiments, flushing fluid is delivered towards optical assembly 212 from both sideholes 312 and from the distal end 304 of a delivery catheter 300 as described herein. Sideholes 312 can be constructed and arranged to allow a flushing fluid to pass from within shaft 302 and through the sideholes 312). Delivery of flushing fluid through sideholes 312 and/or the distal end of the delivery catheter 300 can be performed to clear blood from an area of a luminal segment surrounding optical assembly 212, such as during collecting of image data.

In some embodiments, the delivery of flushing fluid 108 during a flushing procedure is based on a parameter selected from the group consisting of: a pre-determined volume of flushing fluid 108 to be delivered; a pre-determined time during which flushing fluid 108 is delivered; an amount of time of delivery including a time extending from a time prior to retraction of rotating optical core 210 that continued until the collecting of the image data has been completed (e.g. completion of retraction of rotational optical core 210); and combinations of one or more of these. In some embodiments, injector 106 delivers fluid in a flushing procedure with an approximate flow profile selected from the group consisting of: contrast (e.g. between 20% and 100% contrast that can be mixed with saline) at 5 mL s⁻¹ for 6 seconds (e.g. for imaging of a carotid artery including 4 seconds of collecting image data); contrast (e.g. between 20% and 100% contrast that can be mixed with saline) at 4 mL s⁻¹ for 6 seconds (e.g. for imaging of a vertebral artery including 4 seconds of collecting image data); and combinations of one or more of these. In some embodiments, a flushing procedure comprises delivery of flushing fluid 108 (e.g. via one or more delivery catheters 300) for between 2 seconds to 10 seconds, such as a delivery of flushing fluid 108 for approximately 4 seconds (e.g. to purge blood or other non-transparent fluid from a luminal segment of a blood vessel or other area surrounding optical assembly 212 during collection of image data from a patient site). In similar flushing procedures, flushing fluid 108 is delivered at a rate between 3 mL s⁻¹ and 6 mL s⁻¹ (e.g. via one or more delivery catheters 300), to purge non-transparent material.

In these flushing procedures, flushing fluid 108 can comprise a transparent fluid selected from the group consisting of: saline; contrast; dextran; and combinations of one or more of these. In some embodiments, the volume of flushing fluid 108 delivered and/or the time of flushing fluid 108 delivery during a flushing procedure is determined by a parameter selected from the group consisting of: type of procedure being performed; diameter of vessel in which optical assembly 212 is positioned; length of pullback; duration of pullback; and combinations of one or more of these. In some embodiments, flushing fluid 108 is delivered during a flushing procedure by a delivery catheter with an ID greater than 0.027″ (e.g. a first delivery catheter 300a whose distal end 304 is more proximal than a second delivery catheter 300b inserted into the first delivery catheter 300a). In some embodiments, flushing fluid 108 is delivered via multiple lumens 310 in associated multiple delivery catheters 300 (e.g. in the space between two or more pairs of delivery catheters 300 arranged in columnal fashion).

In some embodiments, flushing fluid 108 comprises a first fluid delivered in a first portion of a flushing procedure (e.g. a fluid comprising saline and/or a fluid comprising no or minimal contrast), and a second fluid including contrast (e.g. a second fluid comprising saline and contrast), such as to limit the amount of contrast delivered to the patient during the flush procedure. In these embodiments, injector 106 can comprise two reservoirs (not shown), such as a first reservoir for supplying the first fluid and a second reservoir for supplying the second fluid. When comprised of two reservoirs, injector 106 can be configured to deliver the fluids in each reservoir at different rates, such as to achieve different pressures and/or to provide flushing through different catheters with different IDs. In some embodiments, flushing fluid 108 may be mixed as a solution in a syringe body.

As described herein, optical assembly 212 can be rotated during collection of image data, such as a rotation combined with retraction to create a 3D image of a patient site. In some embodiments, optical assembly 212 is rotated at a rate between 40 rps and 1000 rps, such as a rate of approximately 400 rps. In some embodiments, optical assembly 212 is rotated at a first rate during an imaging mode, and a second rate during a preview mode. In some embodiments, the retraction of optical assembly 212 spans a distance of between 1 cm and 20 cm, such as a retraction of approximately 10 cm. In some embodiments, optical assembly 212 is retracted at a rate of between 1 mm/sec and 200 mm/sec. In some embodiments, the retraction of optical assembly 212 comprises a retraction of approximately 10 cm over 2 seconds and/or a retraction rate of approximately 50 mm/sec. In some embodiments, retraction of optical assembly 212 comprises a resolution of between 5 μm and 20 μm axially and/or a resolution between 20 μm and 50 μm longitudinally. The longitudinal resolution is governed by two factors: the spot-size (light beam cross-section) at the tissue surface being imaged and the spacing between successive rotations of optical assembly 212 during retraction. For a rotation rate of 400 rps and a pullback rate of 50 mm/sec, a pitch of 125 μm between rotations results. In these configurations, a spot size between 20 μm and 40 μm would result in collecting image data which under-samples the objects being imaged. System 103 can be configured to more closely match spot size with pitch, such as by correlating spot size with rotation rate and/or pullback rate.

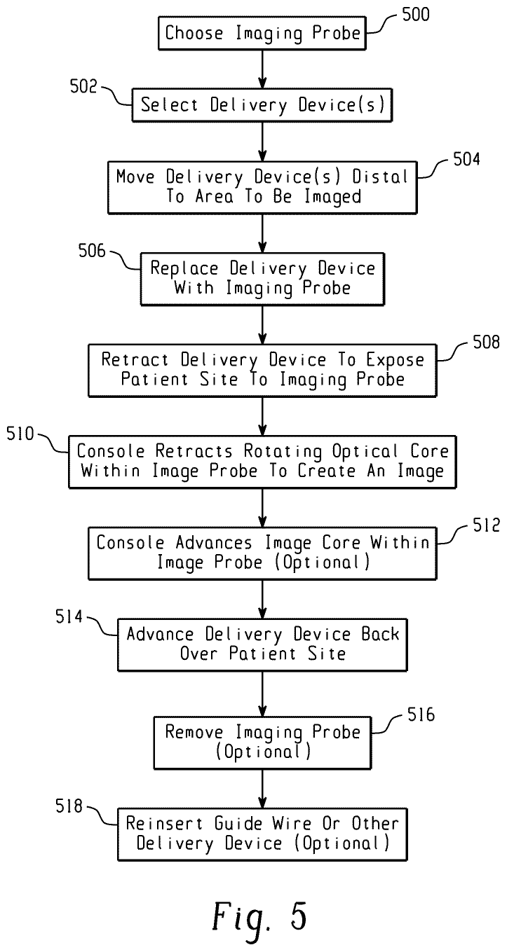

Referring now to FIG. 5, a flow chart of a method of creating an image is illustrated, consistent with the present disclosed concepts. The method of FIG. 5 will be described using the devices and components of system 103 described hereabove in reference to FIG. 1 and further illustrated inf FIGS. 2-4. In Step 1 500, an imaging probe 100 is selected for use. In Step 2 502, at least one delivery device is selected, such as the selection of one or more guidewires 402g and/or delivery catheters 300 described herein. In Step 3 504, a delivery catheter 300 is advanced to a location distal to a patient site, such as a neural site. Step 3 504 can involve the advancement of multiple guidewires 402g and/or delivery catheters 300 each of which are advanced and/or retracted in sequential steps such that a most distal guidewire 402g and/or delivery catheter 300 eventually provides access to a patient site to be imaged (e.g. an intracranial location proximate the patient's brain), from a remote or otherwise different location, such as from the patient's leg (e.g. via a femoral artery), arm (e.g. via the brachial artery or radial artery), or neck (e.g. via a carotid artery). In some embodiments, an anti-coagulation procedure (e.g. the systemic delivery of a blood thinner such as heparin) is performed prior to inserting one or more delivery catheters 300. In some embodiments, one or more guidewires 402g, one or more delivery catheters 300 and/or probe 200 is advanced through one or more veins of the patient. In some embodiments, one or more guidewires 402g, one or more delivery catheters 300 and/or probe 200 is advanced through the spine of the patient (e.g. within the epidural space or intrathecal space of the spine). In some embodiments, one or more delivery devices (e.g. one or more guidewires 402g and/or delivery catheters 300) are advanced to a location within a patient site as described herein in reference to FIGS. 9-29

In Step 4 506, smallest guidewire 402g may be removed and probe 200 is advanced over a guidewire 402g and/or through a delivery catheter 300 to the patient site (e.g. through the smallest diameter delivery catheter 300 of a series of delivery catheters 300 used to access the patient site as described herein).

In Step 5 508, the delivery catheter 300 is withdrawn to expose the patient site to be imaged. In Step 6 510 the optical assembly 212 can be retracted by retracting rotating connector 120 moved by the bedside unit 122. The shaft 202 of probe 200 is kept in place and image data is collected during rotating optical core retraction (e.g. image data used to create a three-dimensional image of tissue proximate the patient site). In some embodiments, optical assembly 212 is positioned (in Step 4 506) distal to the distal end of a delivery catheter 300, and image data is collected while optical assembly 212 does not translate within probe shaft 202. In Step 7 512, in some embodiments, the bedside unit may advance the optical assembly 212 inside the imaging probe 100. This step is optional. The advancement of the optical assembly may also be done when the entire imaging catheter is outside of the body. This is useful if the vessel tortuosity is such that the optical assembly would have difficulty being advanced inside the body. In Step 8 514, optionally, the delivery catheter 300 is advanced over the optical probe 200 to return to the location it had before imaging the patient site. Thus, distal access is maintained. In Step 9 516, optionally, in some embodiments, the imaging probe is removed. In Step 10 518, optionally, a guidewire 402g is inserted (e.g. reinserted) into a delivery catheter 300 after optical probe 200 is removed from the delivery catheter 300 (e.g. after image data is collected by system 103 during a retraction of optical assembly 212). In these embodiments, probe 200 can be subsequently reinserted into the delivery catheter 300 (e.g. after the guidewire 402g is removed and/or to collect additional image data).

In some embodiments, a first delivery catheter 300a comprises a vascular introducer (e.g. a 7 Fr to 9 Fr introducer) which can be placed through the skin of a patient into a blood vessel (e.g. a vein or artery of the leg, arm or neck as described herein) or other anatomical location using standard percutaneous techniques. A second delivery catheter 300b (e.g. a guide catheter or a catheter with an OD between 5 Fr and 7 Fr) can be inserted through delivery catheter 300a, and advanced to a first anatomical location such as a location over the aortic arch. Delivery catheter 300b can be advanced to the first anatomical location over a guidewire 402ga, such as a hydrophilic guidewire comprising an OD between 0.035″ and 0.038″. Delivery catheter 300b can comprise a straight tip or angled tip guide catheter. In some embodiments, prior to advancement of delivery catheter 300b, a separate delivery catheter 300c is inserted through delivery catheter 300a, such as to effectively direct guidewire 402ga into one or more blood vessels (e.g. when delivery catheter 300b comprises a straight tip guide catheter). In these embodiments, after the guidewire 402ga is advanced into the desired blood vessel, the delivery catheter 300c can be removed and replaced with delivery catheter 300b. The process may be iterated as required.

After delivery catheter 300n is in place, probe 200 can be inserted through delivery catheter 300 and advanced to a patient site to be imaged (e.g. advanced over guidewire 402ga, advanced over a smaller or other different guidewire 402gb-n after guidewire 402ga is removed, or advanced without a guidewire after guidewire 402ga is removed). Subsequently, imaging data can be obtained by rotating and/or retracting optical assembly 212 as described herein. Alternative to inserting probe 200 at this time, a smaller delivery catheter 300 can be inserted into and through delivery catheter 300b, such as a delivery catheter 300c comprising an intermediate catheter and/or a catheter with an ID between 0.053″ and 0.070″. Delivery catheter 300c can be advanced to a location more distal than the distal end 206 of delivery catheter 300b. In some embodiments, guidewire 402ga is replaced with a different guidewire 402g, such as a replacement with a smaller guidewire 402gb (e.g. comprising an OD between 0.010″ and 0.014″).

After delivery catheter 300c is in place, probe 200 can be inserted through delivery catheter 300c and advanced to a patient site to be imaged (e.g. advanced over guidewire 402ga, advanced over a smaller or other different guidewire 402gb-n after guidewire 402ga is removed, or advanced without a guidewire after guidewire 402ga is removed). Subsequently, imaging data can be obtained by rotating and/or retracting optical assembly 212 as described in Step 6 510 in FIG. 5. As an alternative to inserting probe 200 at this time, a smaller delivery catheter 300 can be inserted into and through delivery catheter 300c, such as a delivery catheter 300d comprising a microcatheter and/or a catheter with an ID between 0.0165″ and 0.027″. Delivery catheter 300d can be advanced to a location more distal than the distal end of delivery catheter 300c. In some embodiments, guidewire 402ga is removed from delivery catheter 300c, and replaced with a smaller guidewire 300gb, over which delivery catheter 300d is advanced. Guidewire 402gb can comprise a guidewire with an OD between 0.010″ and 0.014″.

After delivery catheter 300d is in place, probe 200 can be inserted through delivery catheter 300d and advanced to a patient site to be imaged (e.g. advanced over guidewire 402ga or 402gb, advanced over a smaller or other different guidewire 402gc-n after guidewire 402a or 402b is removed, or advanced without a guidewire after guidewire 402ga or 402gb is removed). Subsequently, imaging data can be obtained by rotating and/or retracting optical assembly 212 as described herein. In an alternative to inserting probe 200 at this time, delivery catheter 300c can be advanced over delivery catheter 300d (e.g. while a guidewire 402g is within delivery catheter 300d), delivery catheter 300d and any inserted guidewires 402g removed, and probe 200 inserted into delivery catheter 300c, as described herein. Subsequently, imaging data can be obtained by rotating and/or retracting optical assembly 212 as described herein.

Referring now to FIG. 2, a side, partial sectional view of a system comprising an imaging probe is illustrated, wherein the imaging probe comprises an optical assembly configured to collect image data while positioned outside the delivery catheter, consistent with the present disclosed concepts. Imaging probe 100 can comprise probe 200 and at least one delivery catheter 300, each of which can be of similar construction and arrangement to the similar components described hereabove in reference to FIGS. 2, 3 and 4. Probe 200 can comprise proximal connector 208 and flexible spring tip 222 as described herein. Imaging probe 100 and delivery catheter 300 are constructed and arranged such that optical assembly 212 can be positioned distal to the distal end of shaft 202 of delivery catheter 300. For example, probe 200 can comprise a longer length (e.g. a longer shaft 202 length) than the length of delivery catheter 300, such as when probe 200 comprises a length at least 3 cm longer than the length of delivery catheter 300, such as a length at least 5 cm, 10 cm, 15 cm, or 20 cm longer. In some embodiments, shaft 202 of probe 202 comprises an insertable length of up to 250 cm, and a non-insertable length (i.e. a proximal portion of shaft 202) of approximately 100 cm. In some embodiments, at least the distal portion 206 of shaft 202 comprises an OD of approximately 0.014″, such as when delivery catheter 300 comprises an ID of approximately 0.0165″ (e.g. with an OD of approximately 0.022″). In some embodiments, at least the distal portion 206 of shaft 202 comprises an OD between 0.014″ and 0.017″, such as when delivery catheter 300 comprises an OD of no more than 0.032″. In some embodiments, probe 200 comprises a length of up to 350 cm, such as when delivery catheter 300 comprises a length at least 3 cm shorter than the length of probe 200. The optical core 210 can be retracted without retraction of shaft 202 of probe 200 during collection of image data, at least 1 cm, such as a retraction of at least 2 cm, 5 cm, 10 cm, 15 cm, or 20 cm. System 103 can comprise one or more delivery catheters 300, wherein delivery catheter 300n is the smallest diameter of a set of delivery catheters 300a-n currently positioned within the patient, and into which probe 200 has been inserted.

FIG. 6, presents a view of the arteries of the brain. The cerebral arteries may be accessed anteriorly through the basilar artery or posteriorly through the carotid arteries. FIGS. 2, 3 and 4 show the devices required to image inside cerebral arteries per the present disclosure. Typically, they are inserted percutaneously into a patient either through a femoral artery or radial artery as is consistent with cerebral procedures. System 103 comprises probe 200, and a kit of delivery devices including multiple delivery catheters 300, and one or more guidewires 402g. In some embodiments, system 103 comprises one or more similar components to system 103 described hereabove in reference to FIG. 1. Delivery catheters 300 comprise one or more delivery catheters, such as introducer 306, a first delivery catheter 300b (e.g. a guide catheter as described herein), a second delivery catheter 300c (e.g. an intermediate catheter as described herein), and/or a third delivery catheter 300d (e.g. a microcatheter as described herein), as shown in FIG. 3. Delivery catheter 300a has been introduced into a blood vessel of the patient, such as a femoral artery, brachial artery, radial artery, carotid artery or a vein of the patient, such as by using standard percutaneous techniques to place an elongate device through the patient's skin and into a blood vessel. Guidewire 402g (e.g. a guidewire with an OD between 0.035″ and 0.038″) is positioned such that additional delivery catheters can be delivered using an over-the-wire advancement through one or more blood vessels. With femoral or radial access, delivery catheter 300b has been inserted into and through delivery catheter 300a (e.g. over guidewire 402g), such that its distal end is positioned at a location just distal to or otherwise proximate the aortic arch, such as at a location proximate the intracranial artery (ICA) or vertebral artery. Subsequently, delivery catheter 300c has been inserted into and through delivery catheter 300b (e.g. over guidewire 402g), such that its distal end is advanced to an intravascular location as far distal as the middle cerebral artery 600 (MCA). In some embodiments, the distal end of delivery catheter 300c is positioned within or at least proximate a location selected from the group consisting of: internal ceratoid artery; intracranial internal carotid artery (from the cervical ICA); petrous ICA; proximal cavernous ICA; distal cavernous/clinoidal ICA; supraclinoid ICA; the M1 segment V3-4 junction of the vertebral artery; distal V4; proximal basilar; proximal-mid basilar; mid-basilar; and combinations of one or more of these. In some embodiments, guidewire 402g is replaced with a smaller guidewire 402g (e.g. a guidewire with an OD of between 0.010″ and 0.014″ as described herein). Subsequently, delivery catheter 300d has been inserted into and through second delivery catheter 300c (e.g. over guidewire 402g), such that its distal end is positioned at a location proximate (e.g. just distal to, within and/or just proximal to) a patient site, comprising a neural site, as defined hereabove.

As described herein, guidewire 402g can comprise multiple guidewires (e.g., 402ga-n), such as multiple guidewires with different lengths, diameters and/or stiffnesses, such as when a subsequent delivery catheter 300 is advanced over a more flexible guidewire 402g than a previous delivery catheter 300 (e.g. more flexible guidewires 400g are used to advance smaller delivery catheters 300 to a more distal location). In some embodiments, one or more of delivery catheters 300a-n comprise a Tuohy valve of a connector 314a-n, respectively, on their proximal end, such as to reduce blood leakage from the proximal end of the associated delivery catheter 300. In some embodiments, one or more delivery catheters 300 comprise a port 308 (e.g. port 308 shown on the proximal end of delivery catheter 300b), such as to connect to a source of contrast, flushing and/or other fluids to be delivered via a lumen 310 of the associated delivery catheter 300.

In some embodiments, a terminal delivery catheter 300n is retracted to be proximal to the area to be imaged. The optical assembly 212 remains distal to the area to be imaged, outside of the shaft of terminal delivery catheter 300n while collecting image data (e.g. during retraction of the rotating optical core 210) such as is described herein in reference to FIGS. 9-10.

A flushing procedure (e.g. as described herein) can be performed through any delivery catheter 300, prior to and/or during the collecting of image data by optical assembly 212. In some embodiments, pullback of optical assembly 212 is initiated when adequate clearing is confirmed, such as by analysis of image data collected by optical assembly 212 (e.g. an operator analysis of an image or an automated analysis performed by algorithm 118).

In some embodiments, system 103 comprises probe 200 and one or more delivery devices and/or implants configured to treat a disease or disorder such as stroke and/or to remove thrombus from a blood vessel. In these embodiments, system 103 can comprise probe 200 and one or more components selected from the group consisting of: a delivery catheter 300 comprising a balloon guide catheter of approximately between 8 Fr or 9 Fr; a treatment device 114 (described in reference to FIG. 1) comprising a thrombus extraction device; a delivery catheter 300 comprising a distal portion with an OD of approximately 5 Fr, an ID of approximately 0.054″ and/or a length of approximately 132 cm; a delivery catheter 300 configured to deliver a treatment device 114 comprising a thrombus extraction device, such as a catheter with an ID between approximately 0.021″ and 0.027″; a guidewire such as a Stryker Synchro™ guidewire; and combinations of one or more of these. In some stroke treatment or other thrombus removal applications, system 103 comprises probe 200 and one or more components selected from the group consisting of: a delivery catheter 300 comprising a balloon guide catheter of approximately between 8 Fr and 9 Fr (e.g. a delivery catheter advanced into a proximal vessel such as the proximal internal carotid artery or subclavian artery just proximal to the vertebral artery take-off, which is inflated to prevent antegrade flow); a delivery catheter 300 such as a Stryker Merci Balloon Guide Catheter™ of approximately 9 Fr; a Stryker Flowgate™ balloon guide catheter of approximately 8 Fr; a treatment device 114 comprising a Penumbra ACE™ device or similar; a delivery catheter 300 comprising a Covidien Marksman™ microcatheter with a diameter (e.g. an OD) of approximately 0.027″ or similar; a treatment device 114 comprising a Covidien Solitaire FR™ retriever device or similar; and combinations of one or more of these. In some embodiments, system 103 includes both a treatment device 114 comprising a thrombus removal device (e.g. the Penumbra ACE™ device or similar thrombus removal device) and a delivery catheter 300 configured to remove thrombus. In these embodiments, the treatment device 114 comprising the thrombus removal device is used first, and the delivery catheter 300 is used to remove thrombus if the treatment device 114 is unsuccessful at removing sufficient thrombus. In some stroke treatment or other thrombus removal applications including deployment of a thrombus removal device (e.g. a Penumbra ACE™ or other stent retriever), system 103 comprises probe 200 and one or more components selected from the group consisting of: a first delivery catheter 300a comprising an introducer with an ID between 7 Fr and 9 Fr; a second delivery catheter 300b comprising a guide catheter configured to be slidingly received by the first delivery catheter 300a and comprising an ID between 5 Fr and 7 Fr, an ID of approximately 0.088″, a length of between 80 cm and 90 cm and/or a distal end configured to be positioned proximate the aortic arch; a third delivery catheter 300c comprising a reperfusion catheter configured to be slidingly received by the second delivery catheter and comprising an OD between 3.8 Fr and 5.4 Fr and/or a length between 132 cm and 153 cm; a fourth delivery catheter 300d comprising a microcatheter configured to be slidingly received by the third delivery catheter 300c and comprising an OD of approximately 2.6 Fr and/or a length of approximately 160 cm; an injector 106 comprising a power injector; a treatment device 114 comprising a stent retriever or other thrombus removal device; a Penumbra ACE™ stent retriever; and combinations of one or more of these.

In some embodiments, system 103 comprises probe 200 and one or more delivery devices and/or implants configured to treat a disease or disorder such as an aneurysm. In these embodiments, system 103 can be configured to treat the aneurysm by delivering coils, such as when system 103 comprises probe 200 and one or more components selected from the group consisting of: a delivery catheter 300 comprising an approximately 6 Fr guide catheter such as a 6 Fr Cordis Envoy™ catheter (or similar) and/or a 6 Fr Penumbra Neuron™ catheter (or similar); a delivery catheter 300 comprising a Stryker SL-10™ catheter (or similar); an implant 110 comprising one or more embolization coils such as one or more Target™ embolization coils (or similar); an implant delivery device such as a catheter configured to deliver one or more embolization coils; and combinations of one or more of these.

In some aneurysm treatment applications including delivery of coils, system comprises probe 200 and one or more components selected from the group consisting of: a delivery catheter 300 comprising an approximately 6 Fr guide catheter such as a 6 Fr Cordis Envoy™ catheter and/or a 6 Fr Penumbra Benchmark™ catheter; a delivery catheter 300 comprising a Stryker SL-10™ catheter (or similar); a delivery catheter 300 comprising a Covidien Echelon™ catheter (e.g. Echelon 14™, Echelon 10™, or similar), such as a catheter with a length of approximately 155 cm with a 450 or 90° tip angle; a guidewire 402g comprising a Covidien X-Celerator™ hydrophilic guidewire, a Covidien X-Pedion™ guidewire and/or a Stryker Synchro™ guidewire; one or more embolization coils; and combinations of one or more of these. In some aneurysm treatment applications including delivery of coils, system 103 comprises probe 200 and one or more components selected from the group consisting of: a first delivery catheter 300a comprising an introducer with an ID between 7 Fr and 9 Fr; a second delivery catheter 300b comprising a guide catheter configured to be slidingly received by the first delivery catheter and comprising an ID between 5 Fr and 7 Fr, a Touhy valve and/or a length of approximately 90 cm; a third delivery catheter 300c comprising an intermediate catheter configured to be slidingly received by the second delivery catheter 300b and comprising an OD less than 7 Fr and/or a length of approximately 115 cm; a fourth delivery catheter 300n comprising a microcatheter configured to be slidingly received by the third delivery catheter 300c and comprising an ID of approximately 0.0165″ and/or a length of approximately 150 cm; a first guidewire 402g configured to be slidingly received by the first delivery catheter 300a, the second delivery catheter 300b, the third delivery catheter 300c and/or the fourth delivery catheter 300n and comprising a diameter (e.g. an OD) of approximately 0.014″ and/or a length between 175 cm and 190 cm; injector 106 comprising a power injector; treatment device 114 comprising a coil deployment catheter; one or more coils; and combinations thereof.

Alternatively or additionally, system 103 can be configured to treat an aneurysm by implanting a flow diverter, such as when system 103 comprises probe 200 and one or more components selected from the group consisting of: a delivery catheter 300 comprising a guiding catheter such as a guiding catheter with an ID of approximately 6 Fr and/or a length of approximately 110 cm (e.g. configured to be delivered to a location over the aortic arch); a guidewire 402g such as a guidewire with an OD of approximately 0.035″; a Cook Guidewire™ (or similar); a delivery catheter 300 comprising a catheter with an ID of approximately 0.058″, an OD of less than 7 Fr, and/or a length of approximately 115 cm; a delivery catheter 300 with a distal portion with an OD of approximately 2.7 Fr, an ID of approximately 0.027″ and/or a length between 135 cm and 150 cm; a flow diverter such as a Covidien EV3 Pipeline™ flow diverter (or similar); a delivery catheter 300 comprising a delivery catheter 300 configured to deliver a flow diverter such as a Covidien Excelsior™ XT-27 catheter (or similar); and combinations of one or more of these. In some aneurysm treatment applications including implantation of a flow diverter, system 103 comprises probe 200 and one or more components selected from the group consisting of: a delivery catheter 300 comprising an approximately 5 Fr or 6 Fr sheath such as a 6 Fr Cool Flexor Shuttle™ guiding catheter (e.g. which can be delivered over the aortic arch); a delivery catheter 300 of approximately 115 cm length and/or 0.058″ ID, such as a Covidien EV3™ 5 Fr catheter; a delivery catheter 300 comprising a Covidien Marksman™ 0.027″ catheter; an implant 110 comprising a Covidien EV3 Pipeline™ Flow Diverter (e.g. delivered by the Covidien Marksman™ 0.027″ catheter); a guidewire 402g comprising a Cook™ 0.035″ guidewire, a Covidien X-Celerator™ hydrophilic guidewire, a Covidien X-Pedion™ guidewire and/or a Stryker Synchro™ guidewire; and combinations of one or more of these. In some aneurysm treatment applications including implantation of a flow diverter (e.g. a Pipeline™ or Pipeline Flex™ flow diverter), system 103 comprises probe 200 and one or more components selected from the group consisting of: a first delivery catheter 300a comprising an introducer with an ID between 7 Fr and 9 Fr; a second delivery catheter 300b comprising a guide catheter configured to be slidingly received by the first delivery catheter 300a and comprising an ID between 5 Fr and 7 Fr, a Touhy valve and/or a length of approximately 90 cm; a third delivery catheter 300c comprising an intermediate catheter configured to be slidingly received by the second delivery catheter 300b and comprising an OD less than 7 Fr and/or a length of approximately 115 cm; a fourth delivery catheter 300d comprising a microcatheter configured to be slidingly received by the third delivery catheter 300c and comprising an ID less than 0.027″; a first guidewire 402ga configured to be slidingly received by the first delivery catheter 300a, the second delivery catheter 300b, the third delivery catheter 300c and/or the fourth delivery catheter 300n and comprising a length of between 175 cm and 190 cm; a second guidewire 402gb configured to be slidingly received by the first delivery catheter 300a, the second delivery catheter 300b, the third delivery catheter 300c and/or the fourth delivery catheter 300d and comprising a length of between 175 cm and 190 cm; injector 106 such as a power injector; a flow diverter such as a Pipeline™ flow diverter or a Pipeline Flex™ flow diverter; and combinations of one or more of these.

In some aneurysm treatment applications including implantation of a flow diverter (e.g. a Surpass™ or Surpass Future™ flow diverter), system 103 comprises probe 200 and one or more components selected from the group consisting of: a first delivery catheter 300a comprising an introducer with an ID between 7 Fr and 9 Fr; a second delivery catheter 300b comprising a guide catheter configured to be slidingly received by the first delivery catheter 300a and comprising an ID between 5 Fr and 7 Fr, a Touhy valve and/or a length of approximately 90 cm; a third delivery catheter 300c comprising an intermediate catheter configured to be slidingly received by the second delivery catheter 300b and comprising an OD less than 7 Fr and/or a length of approximately 115 cm; a fourth delivery catheter 300n comprising a microcatheter configured to be slidingly received by the third delivery catheter 300c and comprising a Surpass™ delivery catheter, an OD less than 3.3 Fr or less than 3.7 Fr and/or a length of approximately 135 cm; a first guidewire 402ga configured to be slidingly received by the first delivery catheter 300a, the second delivery catheter 300b, the third delivery catheter 300c and/or the fourth delivery catheter 300n and comprising an exchange length guidewire; injector 106 such as a power injector; a flow diverter such as a Surpass™ flow diverter and/or a Surpass Future™ flow diverter; and combinations of one or more of these.

Alternatively or additionally, system 103 can be configured to treat an aneurysm by delivering stent assisted coils, such as when system 103 comprises probe 200 and one or more components selected from the group consisting of: a delivery catheter 300 comprising an approximately 6 Fr guide catheter such as a 6 Fr Cordis Envoy™ catheter (or similar) and/or a 6 Fr Penumbra Neuron™ catheter (or similar); a delivery catheter 300 comprising a Cordis Prowler Select Plus™ catheter; an implant delivery device 112 and/or implant 110 comprising Cordis Enterprise™ vascular reconstruction device; a delivery catheter 300 comprising a Stryker XT27™ catheter; an implant delivery device 112 and/or implant 110 comprising a Stryker Neuroform EZ™ stent system; an implant 110 comprising one or more stents; an implant 110 comprising one or more embolization coils; and combinations of one or more of these. In some aneurysm treatment applications including delivery of stent assisted coils, system 103 comprises probe 200 and one or more components selected from the group consisting of: a delivery catheter 300 comprising an approximately 6 Fr guide catheter such as a 6 Fr Cordis Envoy™ catheter and/or a 6 Fr Penumbra Neuron™ catheter; a delivery catheter 300 comprising a Cordis Prowler Select™ for Enterprise and/or a Covidien Marksman™ for Neuroform™; an implant 110 and/or delivery device 112 comprising a Stryker Neuroform EZ™ stent system; an implant 110 comprising one or more stents; an implant 110 comprising one or more embolization coils; a guidewire 402g comprising a Covidien X-Celerator™ hydrophilic guidewire, a Covidien X-Pedion™ guidewire and/or a Stryker Synchro™ guidewire; and combinations of one or more of these.

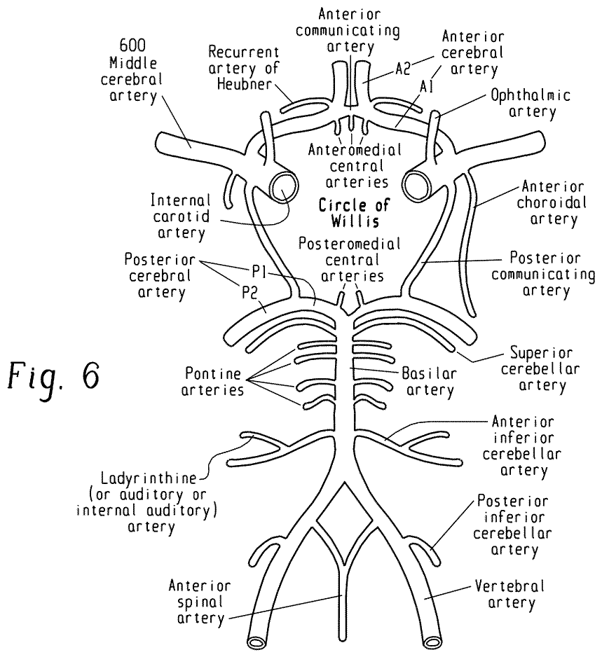

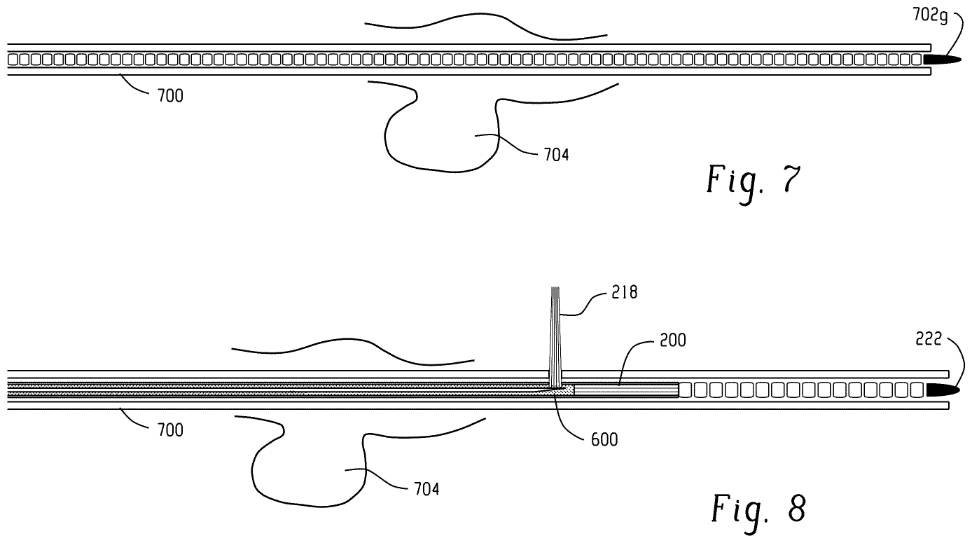

Referring now to FIGS. 7-13, schematic anatomical views of a series of steps for creating an image are illustrated, including advancing an imaging probe beyond the distal end of a delivery catheter prior to collecting image data, consistent with the present disclosed concepts. System 103 includes imaging probe 100 and one or more delivery devices, such as at least one delivery catheter 700 and at least one guidewire 702g. In some embodiments, system 103 comprises one or more similar components to system 103 described hereabove in reference to FIG. 1. In some embodiments, system 103 has been introduced into the patient as described hereabove in reference to FIG. 1, 2-6, such as when delivery catheter 300 of FIG. 3 comprises one or more delivery catheters 300 (e.g., delivery catheters 300a-n), including at least delivery catheter 300a of FIG. 3. In FIG. 7, guidewire 702g has been advanced through a vessel, such as a blood vessel, such that its distal end is at or beyond patient site 704. Delivery catheter 700 has been partially advanced over guidewire 702g.

While patient site 704 of FIGS. 7-13 is shown to include an aneurysm, alternatively or additionally patient site can comprise a site of a different patient disease or disorder, a site including an implant such as implant 110, a site including a patient treatment device such as treatment device 114 and/or any internal body location of the patient.

In FIG. 7, the distal end of delivery catheter 700 and the distal end of guide wire 702g have been advanced distal to patient site 704. In FIG. 8, guidewire 702g has been removed, and probe 200 has been inserted through delivery catheter 700 such that its distal end, comprising flexible spring tip 222 is proximate the distal end of delivery catheter 700. The probe 200 is advanced such that optical assembly 600 is positioned distal to and/or within patient site outside of delivery catheter 700. In FIG. 9 the delivery catheter 700 is retracted to a position proximal to the patient site 704 to be imaged. Subsequently, image data is collected while retracting optical assembly 600 to the position shown in FIG. 10. Shaft 202 of probe 200, analogous to delivery catheter 700, remains stationary during image acquisition. The image data collected can comprise image data of the blood, vessel wall and other tissue within patient site and/or image data of occlusive matter (e.g. thrombus or plaque) within patient site. In some embodiments, the image data further includes image data of an implant (e.g. implant 110 described herein), and/or image data of a treatment device such as treatment device 114.

A flushing procedure (e.g. as described herein) can be performed through any delivery catheter 300, during one or more of Steps 3-10 (504-518) as described in FIG. 5. In some embodiments, a flushing procedure is at least performed during Step 6 510, such as via delivery catheter 700 shown in FIG. 9, or a more proximal delivery catheter (not shown). In some embodiments, pullback of optical assembly 600 is initiated when adequate clearing is confirmed by analysis of image data collected by optical assembly 600 (e.g. an operator analysis of an image or an automated analysis performed by algorithm 118).

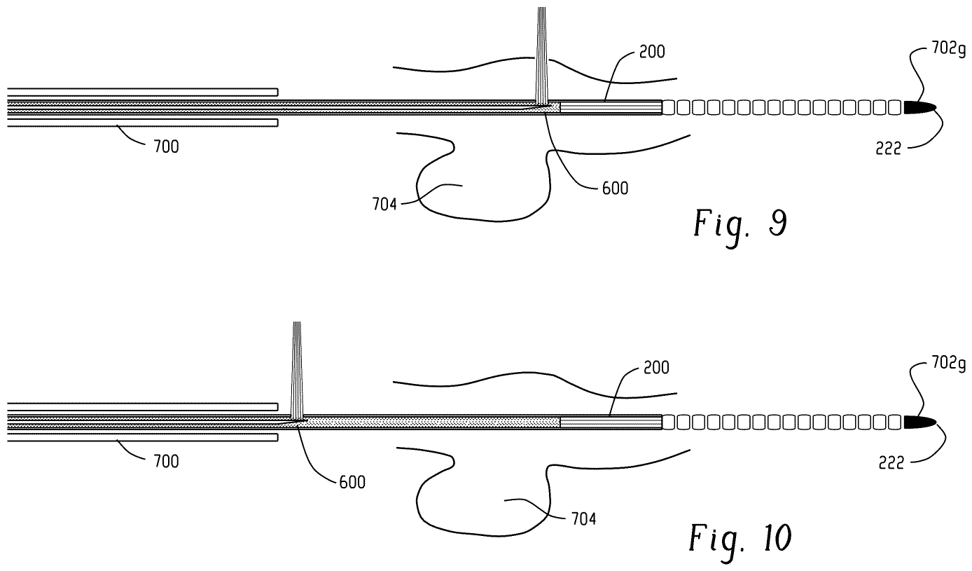

FIG. 11 is an illustration of Step 7 512 of the method of FIG. 5 The optical assembly 600 is advanced through the patient site 704

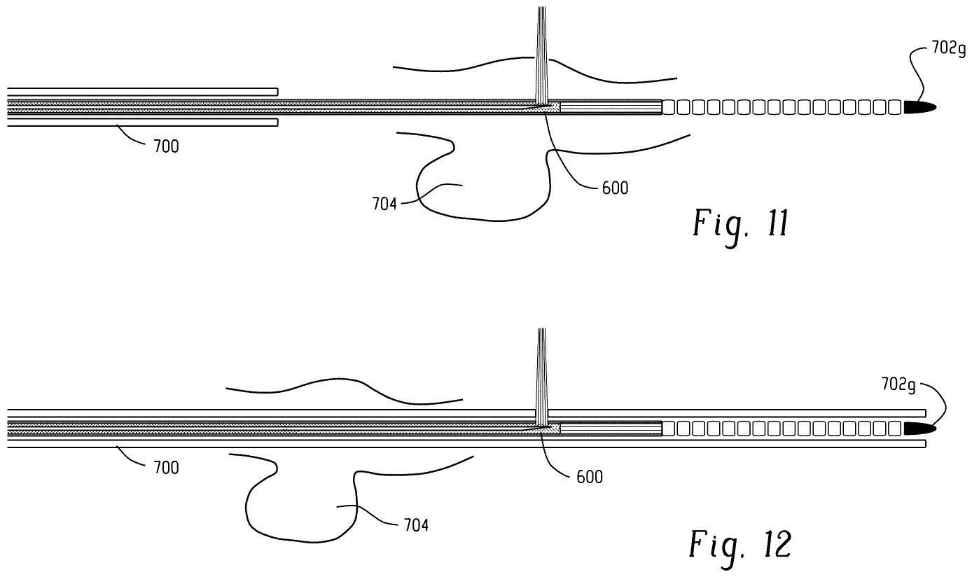

FIG. 12 is an illustration of Step 8 514 of the method of FIG. 5 The optical assembly 600 is now past patient site 704 and delivery catheter 700 is advanced over probe 200.

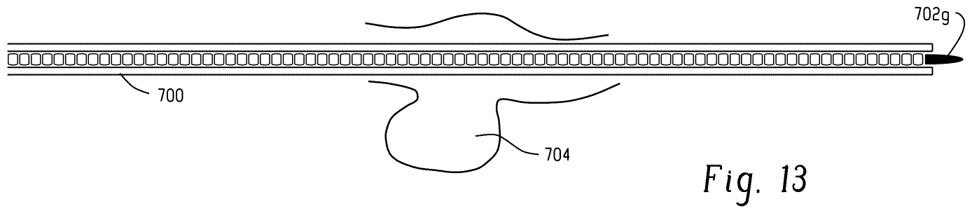

Referring now to FIG. 13, delivery catheter 700 is advanced along guidewire 702g past patient site 704. While optical assembly 600 is removed. Advancement of the delivery catheter 700 is the key to maintaining access to the patient site 704.

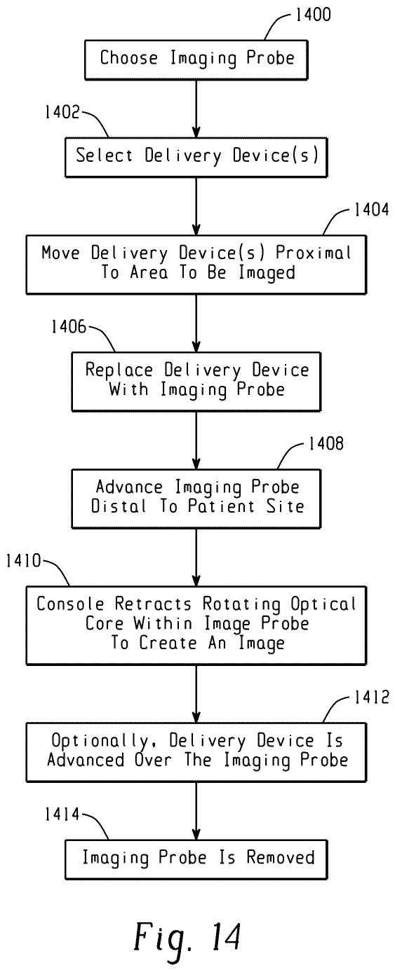

FIG. 14 presents a series of steps for creating an image, including keeping the delivery catheter proximal the patient site and the optical assembly of an imaging probe distal to the patient site prior to collecting image data, consistent with the present disclosed concepts. Seen in step 1 1400, an imaging probe is chosen. In step 2 1402, one or more delivery devices are selected. For step 3 1404 the delivery devices may be moved or positioned proximal to the area to be imaged. In step 4 1406, the delivery device is replaced with an imaging probe. In step 5 1408, the imaging probe is advanced through and/or past a patient site to a distal position. In step 6 1410 a console may retract a rotating optical core within the imaging probe to create an image. In step 7 1412, the delivery device may optionally or instead of be advanced over the imaging probe into position within a patient site. Finally in step 8 1414, the imaging probe is removed.

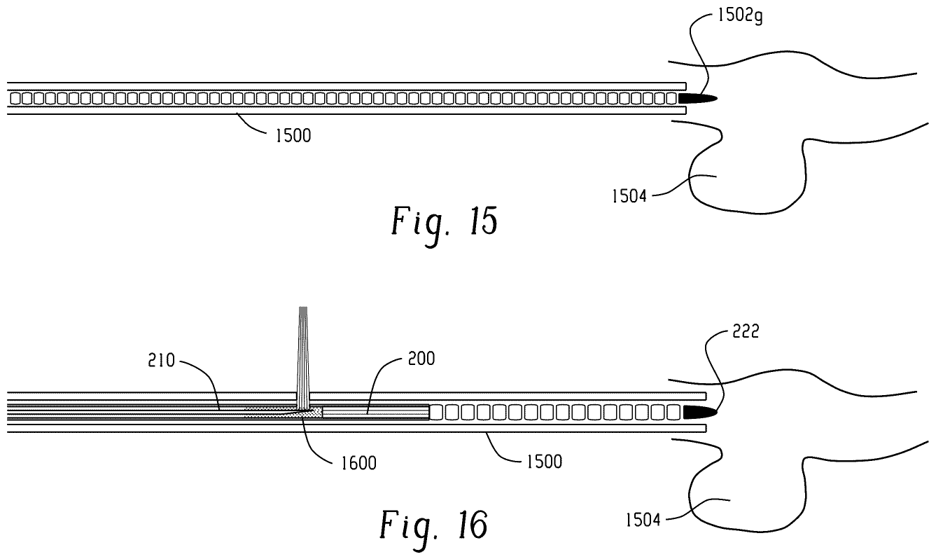

An illustration of the above-described method is presented in FIGS. 15-18. In FIG. 15, guidewire 1502 has been advanced through a vessel, such as a blood vessel, such that its distal end is at or proximal to patient site 1504. Delivery catheter 1500 has been advanced over guidewire 1502. While patient site 1504 of FIGS. 15-18 is shown to include an aneurysm, alternatively or additionally patient site can comprise a site of a different patient disease or disorder, a site including an implant such as implant 110, a site including a patient treatment device such as treatment device 114 and/or any internal body location of the patient such as those described herein.

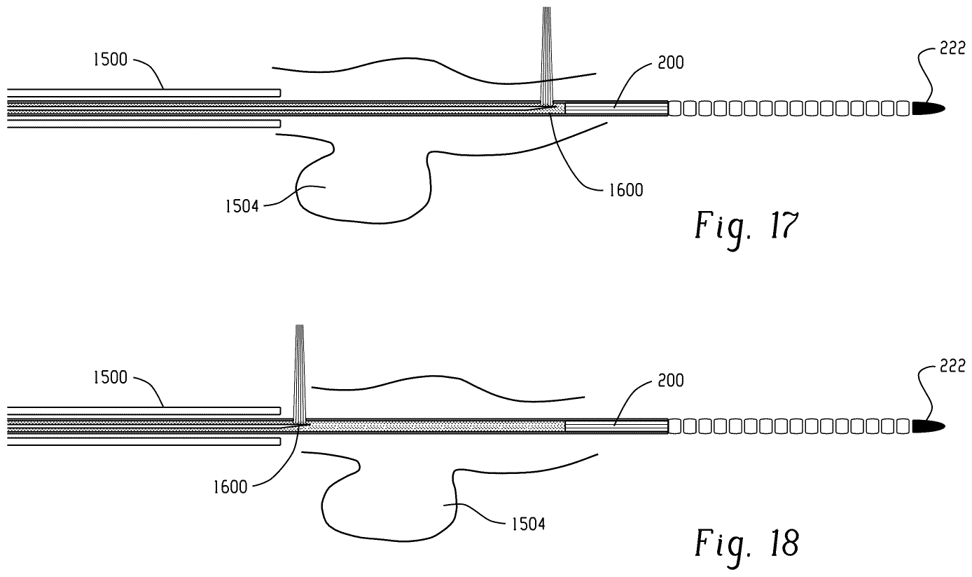

In FIG. 16, guidewire 1502g has been removed, and probe 200 has been inserted through delivery catheter 1500 such that its distal end, comprising flexible tip 222 is proximate the distal end of delivery catheter 1500. In FIG. 17, the probe 200 is positioned such that optical assembly 1600 is distal to and/or within patient site, outside of delivery catheter 1500. Subsequently, image data is collected while retracting shaft rotating optical core 210 of probe 200 to the position shown in FIG. 18. The image data collected comprising image data of the blood, vessel wall and other tissue within patient site and/or image data of occlusive matter (e.g. thrombus or plaque) within patient site. In some embodiments, the image data further includes image data of an implant (e.g. implant 110 described herein, and/or image data of a treatment device such as treatment device 114).

A flushing procedure (e.g. as described herein) can be performed through any delivery catheter 300, during one or more of Steps 3-7 (1404-1412) in FIG. 14. In some embodiments, a flushing procedure is at least performed during Step 6 1410, such as via delivery catheter 1500 shown, or a more proximal delivery catheter (not shown). In some embodiments, pullback of optical assembly 1600 is initiated when adequate clearing is confirmed by analysis of image data collected by optical assembly 1600 (e.g. an operator analysis of an image or an automated analysis performed by algorithm 118).

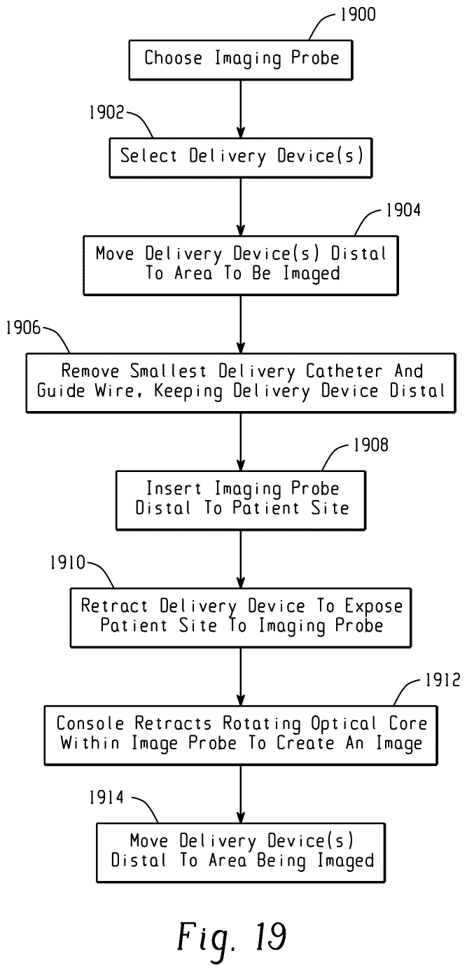

FIG. 19 is a flow chart of a method to create an image with the smallest catheter removed. In step 1 1900, and imaging probe is chosen. In step 2 1902, one or more delivery devices are chosen. In step 3 1904, the delivery device is moved distal to an area to be imaged. In step 4 1906, the smallest delivery catheter and guide wire are removed while keeping the delivery device distal. In step 5 1908, an imaging probe is inserted and positioned distal to the patient site. In step 6 1910, the delivery device is retracted to expose the patient site to the imaging probe. In step 7 1912, a console can retract a rotating optical core within the image probe to create an image of the patient site. In step 8 1914, the delivery device is again moved distal to the imaged area.

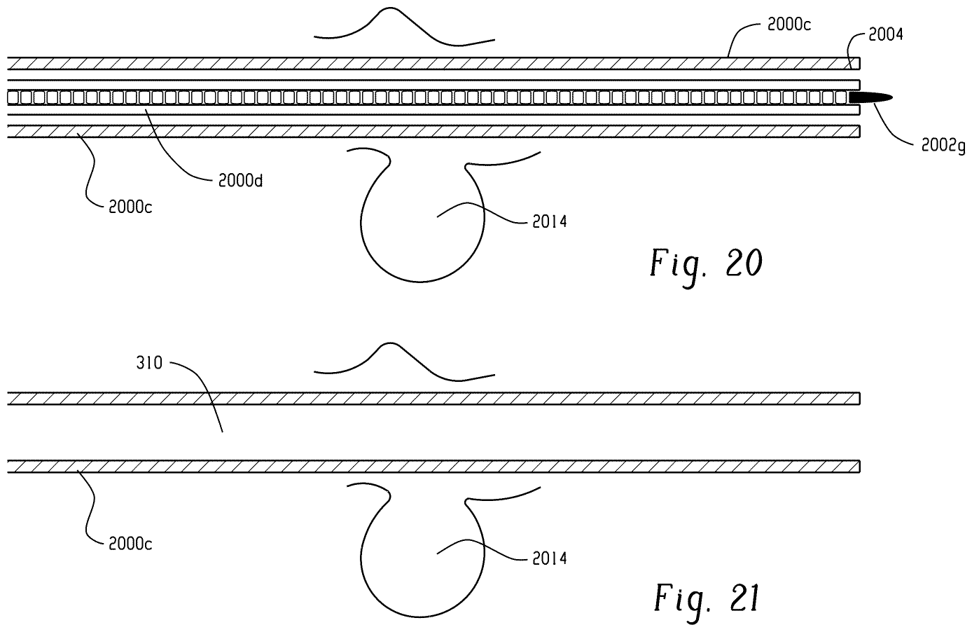

Referring now to FIGS. 20-24, a delivery catheter 2000c is advanced over a smaller delivery catheter 2000d, after which the smaller delivery catheter 2000d and guidewire 2002g are removed from the lumen 310 of the larger delivery catheter 2000c and probe 200 inserted in its place. System 103 includes imaging probe 100, one or more delivery devices, such as at least one delivery catheter 2000c and at least one guidewire 2002g. As shown in FIG. 20, delivery catheter 2000d has been inserted into the larger delivery catheter 2000c. (e.g. over guidewire 2002g with an OD of between 0.010″ and 0.014″). Delivery catheter 2000d has been advanced such that distal end of delivery catheter 2000d has passed through and proximate to the distal end 2004 of delivery catheter 2000c. (for example, such that distal end 2004 is positioned within and/or beyond patient site 2014). While patient site 2014 of FIGS. 20-23 is shown to include an aneurysm, alternatively or additionally patient site can comprise a site of a different patient disease or disorder, a site including an implant such as implant 110, a site including a patient treatment device such as treatment device 114 and/or any internal body location of the patient such as those described herein.

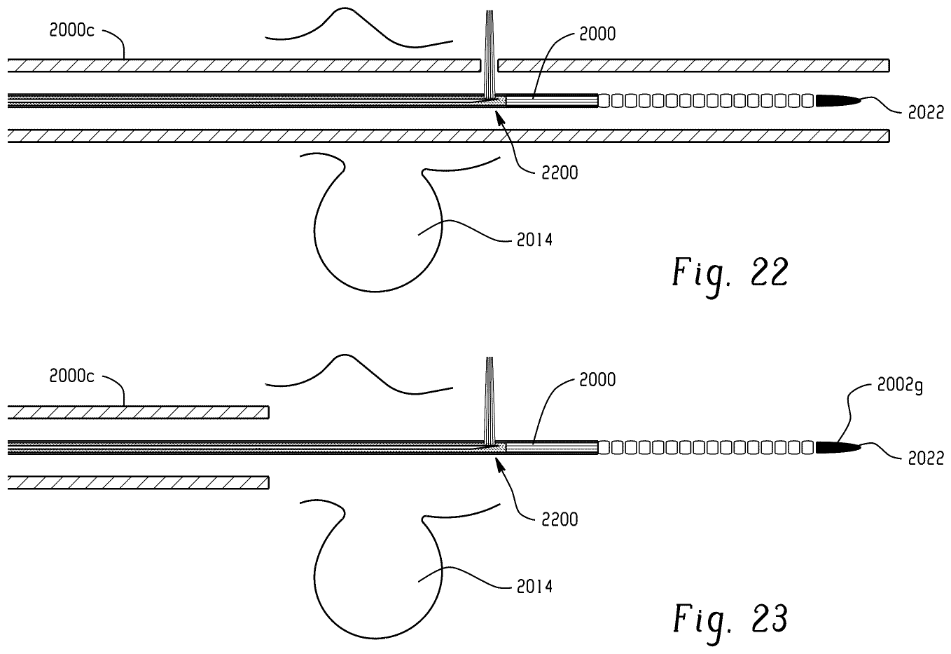

Delivery catheter 2000c can have already been inserted (as described stepwise in FIG. 19) through one or more other delivery catheters 2000, such as when delivery catheter 2000c is inserted through a larger delivery catheter 2000b (not shown), which has previously been inserted through a delivery catheter comprising an introducer (e.g. a vascular introducer, not shown). As shown in FIG. 20 in certain embodiments, delivery catheter 2000c is subsequently advanced over a smaller delivery catheter so it is distal to the patent site, (e.g. when delivery catheter 2000d is positioned over a guidewire 2002g distal to the patent site), as shown in in FIG. 20. This subsequent advancement of the larger delivery catheter 2000c over delivery catheter 2000d can provide a safer and/or more effective advancement of the delivery catheter 2000c than would have been accomplished by advancing the delivery catheter 2000c over guidewire 2002g alone (without the benefit of support provided by delivery catheter 2000d). The distal end 2004 of delivery catheter 2000c can be positioned at a location distal to patient site 2014 (as shown), within patient site 2014 and/or proximal to patient site 2014. After the advancement of the delivery catheter 2000c over the delivery catheter 2000d, the smaller delivery catheter 2000d, and if appropriate, the surrounded guidewire 2002g, can be removed, as shown in FIG. 21. Subsequently, probe 2000 can be advanced through delivery catheter 2000c, as shown in FIG. 22, such as to a location within and/or just distal to patient site 2014 (e.g. optical assembly 2200 is positioned within and/or just distal to patient site 2014). The removal of delivery catheter 2000d can be performed to accommodate a larger diameter probe 2000 and/or to provide a larger space between probe 2000 and the surrounding delivery catheter 2000c (e.g. to reduce the resistance of flushing fluid 108 as described herein).

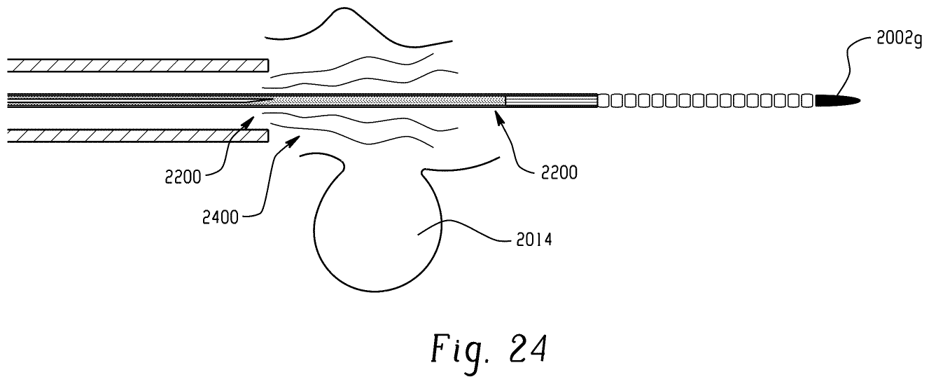

In some embodiments, the probe 2000 comprises an OD between 0.014″ and 0.025″, and the surrounding delivery catheter 2000c comprises an ID between 0.053″ and 0.070″. As shown in FIG. 23 delivery catheter 2000c can be retracted. As shown in FIG. 24 the rotating optical core can be retracted to after a flushing procedure has been initiated, and image data collected as optical assembly 2200 translates to the proximal end of patient site 2014, as shown in FIG. 24. Flushing fluid 2400 (e.g. flushing fluid 2400 via injector 106) can be delivered in the space between probe 200 and delivery catheter 2000 c as illustrated in FIG. 24. Alternatively, or additionally, flushing fluid 2400 can be delivered in the space between any two delivery catheters (e.g. in the space between delivery catheter 2000b and 2000c, and/or in the space between 2000a and 2000b), via the delivery catheters distal end 2004 or more side holes 312 described herein. Additional or fewer delivery catheters may be used.

As shown in FIG. 22 the delivery catheter 2000c can be readvanced over probe 2022. Then as shown in FIG. 21, the imaging probe can then be removed. Then as shown in FIG. 20 the guidewire 2002g and delivery catheter 2000d can be readvanced. Thus, distal access is maintained.

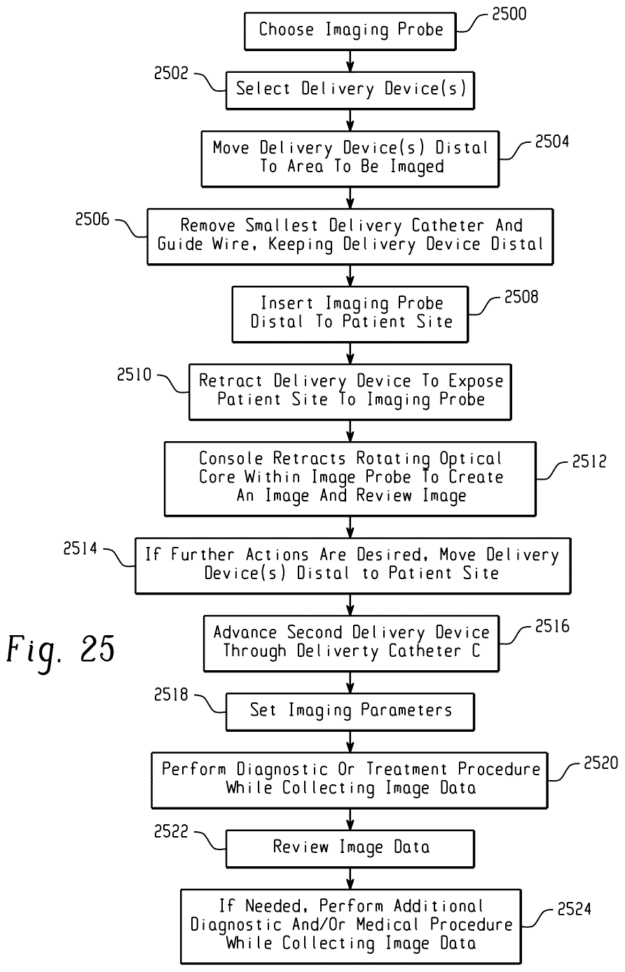

FIG. 25 presents a flow chart of a method of creating an image using an imaging probe in a side-by-side configuration with a second device is illustrated, consistent with the present disclosed concepts. In Step 1 2500 an imaging probe is selected. In Step 2 2502 delivery devices 2000 (consistent with catheters 300a-n) are selected. These may include delivery catheter 2000c with an ID of between 0.0165″ and 0.027″. It is advanced over a guidewire 2002g to a location proximate a patient site (e.g. a neural site as described herein). In some embodiments, guidewire 2002g is delivered distal to patient site, such as at a location just distal to an aneurysm neck or just distal to thrombus or other occlusive matter. Delivery catheter 2000d and guidewire 2002g can be inserted through one or more, larger delivery catheters, such as delivery through an introducer catheter such as delivery catheter 2000a described herein. In Step 3 2504, delivery catheter 2000d and guidewire 2002g are moved distal to patient site. In Step 4 2506 guidewire 2002g and smallest delivery catheter, such as catheter 300n, are removed. (e.g. removed from a lumen of delivery catheter 2000c). In Step 5 2508, the imaging probe is delivered through delivery catheter 2000c. In Step 6, delivery catheter 2000c is moved proximal to the patient site 2014. In Step 7 2512, a console retracts rotating image core to create an image, and the generated image is reviewed either by an operator or algorithmically. In certain embodiments the console may tell a retracting assembly 124 to retract inside bedside unit 122. In Step 8 2514, if image review justifies further action, delivery device 2000c is inserted past patient site over imaging catheter. In Step 9 2516, a second delivery device for treatment or a diagnostic catheter is inserted thru delivery catheter 2000c proximate to the patient site. Delivery catheter 2000c can comprise a delivery catheter with an ID of between 0.053″ and 0.085″. In Step 10 2518, imaging parameters are set. These may include a continuous scan of the treatment/diagnostic device during deployment as the bedside unit advances and retracts rotating image core 210 inside of stationary image catheter shaft 202. It also may include a manual advance and retract mode. Image data may be configured to be captured continuously or intermittently. In Step 12 2522 imaging data is once again reviewed. In Step 13 2524 additional diagnostic or treatment procedures are performed if deemed necessary and optionally additional imaging can occur. If additional diagnosis and/or treatment is not desired, the devices are removed from the patient, and the procedure is complete. Additional or fewer delivery catheters may be used.

During any time in the procedure, System 103 can be configured to deliver flush material during image data capture, such as flushing fluid 108 delivered via injector 106 as described herein. In some embodiments, console 104 is set in a continuous image data capture mode, and flushing fluid 108 can be delivered for approximately 20 seconds at a flow rate of between 2 mL s⁻¹ and 3 mL s⁻¹. In some embodiments, console 104 is set in an intermittent data capture mode, and flushing fluid 108 can have a delivery profile approximating flushing every 10 seconds for a 2 second duration, such as when flushing fluid 108 is delivered at 3 mL s⁻¹ to 4 mL s⁻¹. In some embodiments, console 104 is configured to automatically detect delivery of flushing fluid 108. In some embodiments, console 104 is configured to control the delivery of flushing fluid 108 via injector 106, such as an automatic, semi-automatic and/or manual control as described hereabove. The flushing procedure can be performed through any delivery catheter 300, during one or more of steps of this method of FIG. 25.

In some embodiments, treatment/diagnostic delivery device 136 comprises a catheter configured to deliver one or more coils (e.g. occlusive coils configured to treat an aneurysm). In some embodiments, treatment device 114 comprises a stentriever or other thrombus removal device. In some embodiments, treatment/diagnostic delivery device 136 comprises a stent delivery device, such as a covered stent delivery device.

During any time in the procedure images may be assessed by reviewing of a 2D and/or 3D image of any implants (e.g. one or more implants 110) implanted in the patient during the procedure, such as to determine sufficient occlusion (e.g. sufficient occlusion of an aneurysm by implantation of coils or a covered stent), adequate positioning and/or apposition of an implant with tissue, adequate flow through a native vessel, and combinations of one or more of these.

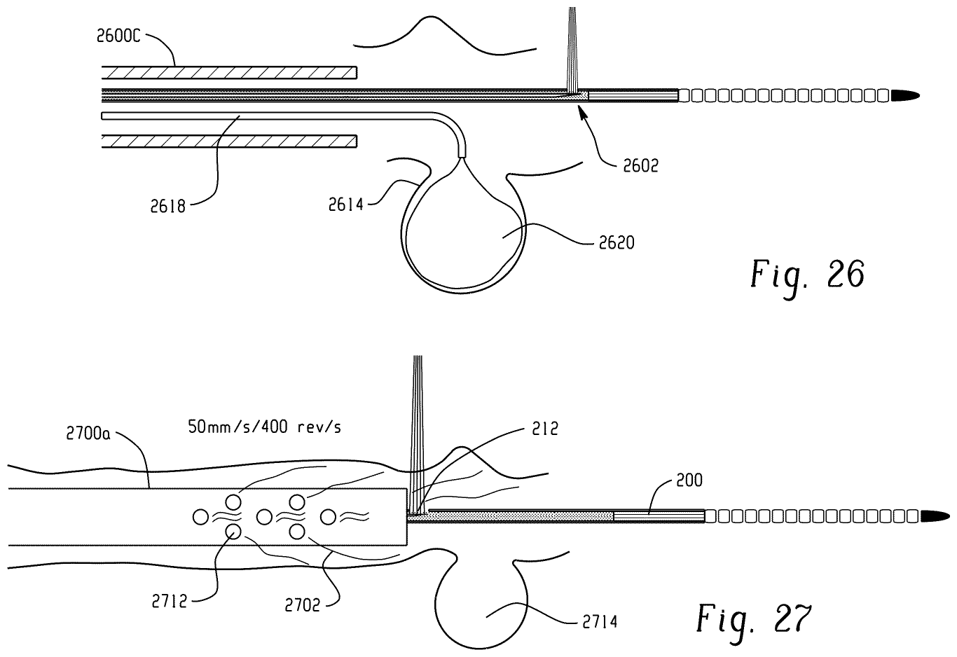

Referring now to FIG. 26, a schematic anatomical view of an imaging probe in a side-by-side configuration with a second device is illustrated, consistent with the present disclosed concepts. In line with a configuration as presented in FIG. 1, system 103 includes imaging probe 100, one or more delivery devices, such as at least one delivery catheter 300 and at least one guidewire 102 (not shown but such as is described in reference to FIG. 25), and a device to treat and/or diagnose the patient, such as implant delivery device or treatment device 114. Treatment/diagnostic delivery device 136 can be configured to deliver implant (e.g. a coil as shown, a stent and/or a covered stent). FIG. 26 comprises one or more delivery catheters 2600a-n, such as one or more delivery catheters 2600c including at least a delivery catheter analogous to delivery catheter 300a of FIG. 3. Delivery catheter 2600c comprises shaft 302 and lumen 310 as illustrated in FIG. 3. In some embodiments, system 103 has been introduced into the patient as described in reference to FIG. 25, such as when imaging probe 100 and treatment/diagnostic delivery device 2618 are in the side-by-side configuration within lumen 310 as shown in FIG. 26. Optical assembly 2602 and the distal end of device are each positioned proximate patient site, such that a diagnostic procedure (e.g. a biopsy) and/or a treatment procedure (e.g. placement of a coil or other implant 110) can be performed using treatment/diagnostic delivery device 2618, with imaging probe optical assembly 2602 collecting image data prior to, during and/or after the diagnosis and/or treatment by treatment/diagnostic delivery device 2618. For example, a hardening gel 2620 is seen leaving diagnostic delivery device 2618 to fill the patient site 2614 shown as an aneurism.

The distal end of all the catheters shown in FIGS. 2 & 3 in some embodiments, may include a functional element 214, 316 that can comprise a visualizable marker, such as a marker selected from the group consisting of: a radiopaque marker; an ultrasonically reflective marker; a magnetic marker; a metallic spring tip, a polymer tip, a ferrous marker; and combinations of one or more of these. In some embodiments, delivery catheter 300 comprises a connector 318 on its proximal end, such as a Touhy or other connector comprising a port (e.g. port 308 described herein) which can attach to a supply of fluids as described herein (e.g. a syringe, a power injector, and the like).

Referring now to FIG. 27, a schematic view of a system comprising an imaging probe and a delivery catheter including side holes delivering a flushing fluid 108 is illustrated, consistent with the present disclosed concepts. Seen in conjunction with FIG. 1, System 103 comprises probe 200 and at least one delivery catheter 2700a, each of which can be of similar construction and arrangement to the similar components described hereabove in reference to FIGS. 2,3 & 4. Delivery catheter 2700a and probe 200 have been inserted into the patient (such as in an interventional procedure including one or more delivery catheters 2700 as described herein in reference to FIGS. 2,3 & 4, 5-26). Shaft 202 of probe 200 is advanced such that optical assembly 212 is positioned within or just distal to patient site 2714. Patient site can comprise an aneurysm, stenotic location, an implant location, a treatment device location and/or other patient site as described herein. In some embodiments, optical assembly 212 is positioned distal to the distal end of delivery catheter 2600, as shown in FIG. 26 (such as to perform an image data collecting pullback procedure where optical assembly 2602 remains outside of shaft 202 throughout the retraction).

System 103 further comprises injector 106, which can be configured to deliver one or more fluids to one or more delivery catheters 2600 or other components of system 103. The port 308 of delivery catheter is fluidly attached to injector via tubing, such that fluid can be delivered into the lumen 216 of delivery catheter 2600.

Just prior to image data collection, delivery of flushing fluid 108 can be initiated by injector 106 propelling fluid into lumen 310 of delivery catheter 2600, such as to begin a flushing procedure to clear undesired material from locations surrounding optical assembly 2602 (e.g. blood or other material that would prevent or at least limit image data collection by optical assembly 2602). Flushing fluid 108 is delivered to these locations via the distal end 304 of lumen 310 of delivery catheter 2600 and/or via sideholes 2700. During image data collection (e.g. during rotation and retraction of optical assembly 2602), delivery of flushing fluid 108 by injector 106 continues.

In some embodiments, delivery catheter 2600 comprises multiple delivery catheters 300, one or more of which can include sideholes 2712, and each of which can be used to deliver flushing fluid 108 in a flushing or other fluid delivery procedure. Flushing fluid 108 can comprise a contrast material (e.g. a ratio of radiopaque contrast and saline) such as to also allow fluoroscopic imaging of the patient site and neighboring areas.

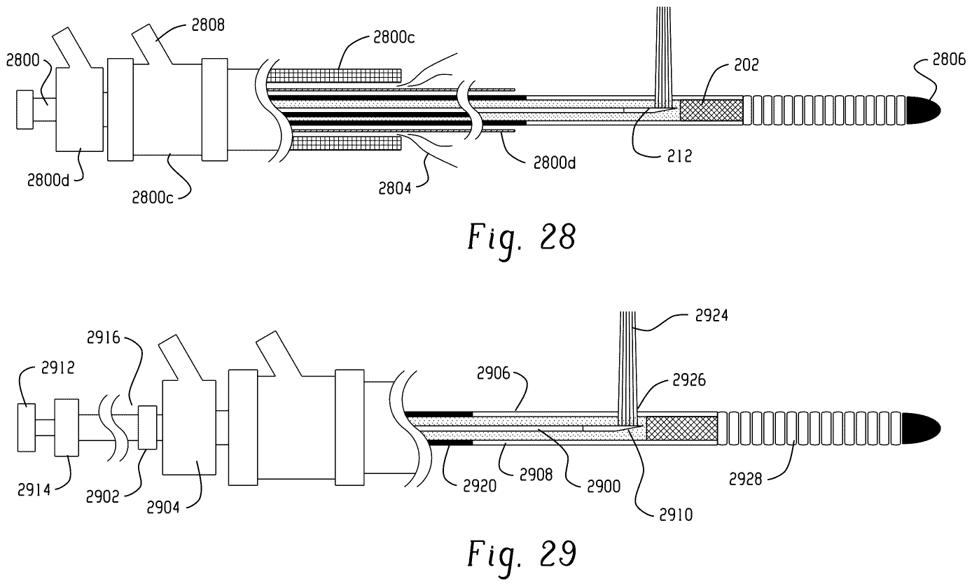

In conjunction with FIGS. 1 and 2 and referring now to FIG. 28 which presents a schematic anatomical view of a system comprising an imaging probe and a delivery catheter including delivering a flushing fluid 108 between delivery catheter 2800c and 2800d, consistent with the present disclosed concepts. System 103 comprises probe 2800 and at least one delivery catheter 2802, each of which can be of similar construction and arrangement to the similar components described hereabove in reference to FIGS. 2-4 and 20-24. Delivery catheter 2802c and probe 2800 have been inserted into the patient (such as in an interventional procedure including one or more delivery catheters 2802 is described hereabove in reference to FIGS. 2 and 5-26). Shaft 202 of probe 2800 is advanced such that optical assembly 212 is positioned within or just distal to a patient site. Patient site can comprise an aneurysm, stenotic location, an implant location, a treatment device location and/or other patient site as described herein.

System 103 further comprises injector 106, which can be configured to deliver one or more fluids to one or more delivery catheters 2802 or other components of system 103. A port of a delivery catheter is fluidly attached to injector via tubing, such that fluid can be delivered in the space of lumen 216 that surrounds shaft 202 of probe 2800 (e.g. the space between the outer wall of shaft 202 and the inner wall of delivery catheter 300).

In conjunction with FIG. 3 and continuing to refer to FIG. 28, flushing fluid 108 may be delivered to these locations via the distal between delivery catheter b and c, (distal end of lumen 310) and/or via sideholes 312. During image data collection (e.g. during rotation and retraction of optical assembly 212), delivery of flushing fluid 108 by injector 106 continues. In some embodiments, a flushing procedure includes delivery of flushing fluid 108 via both the distal end of shaft 302 and sideholes 312. Alternatively, flushing fluid can be delivered between delivery catheter 2802 c and d. Flushing fluid 108 delivery through these larger delivery catheters reduces delivery pressure and allows for higher flushing fluid 108 flow rates.

### Optical Core Description

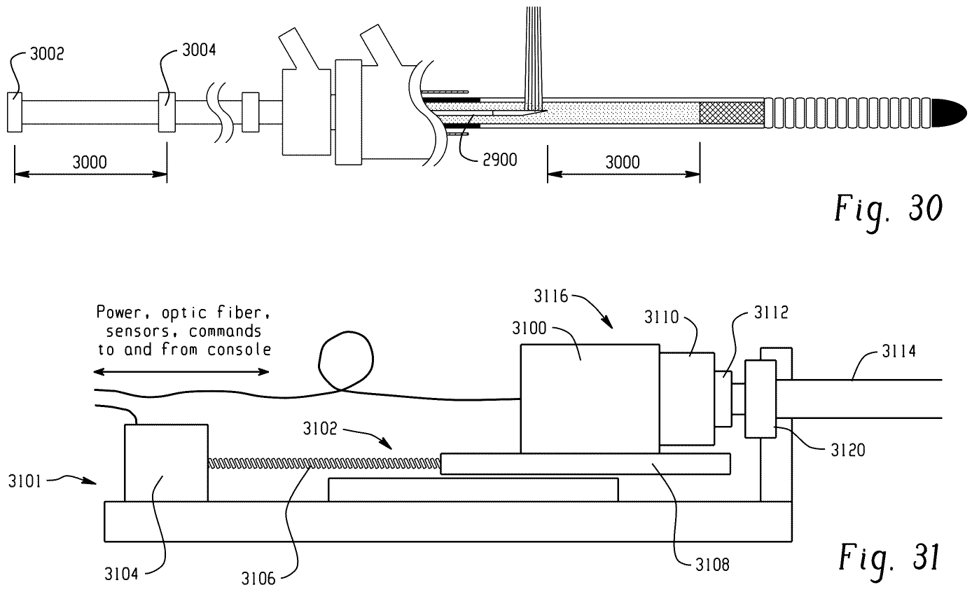

FIG. 29, presents a schematic view of a probe comprising, a rotating optical core 2900 is illustrated with the rotating optical core 2900 in its most distal position, consistent with the present disclosed concepts and more fully described in context with FIGS. 30-34.

Referring now to FIG. 30, is a schematic view of the optical core 2900 translated to a proximal location. Imaging distance 3000 is equal to the translation distance. A bedside unit retracts rotating image core by the imaging distance 3000. The probe shaft has a transparent section 2908 at least as long as the imaging distance 3000.

After creating an image, the connector translating section 3002 translates the rotating optical core distal the imaging distance 3000, replacing it in the most distal location as shown in FIG. 29.

Referring additionally to FIG. 31 console 104 may be attached to a bedside unit 3101. The bedside unit can comprise spin motor 3100 constructed and arranged to rotate at least rotating optical core 2900. Bedside unit 3101 can further comprise a retraction assembly 3102, constructed and arranged to retract rotating optical core 2900. In some embodiments retraction assembly 3102 may consist of a translating motor 3104, lead screw 3106 and a slide 3108. In some embodiments spin motor 3100, fiber optic rotary joint 3110 and connection to connector translating section 3112 may be mounted on retraction assembly 3102.

In some embodiments, retraction assembly 3102 and probe 200 can be configured such that during image data collection, retraction assembly 3102 retracts optical assembly 2910 (shown emitting light 2924) only and not the shaft 3114. In these embodiments, shaft 3114 can comprise a relatively long transparent section 2908, surrounding optical assembly 2910, since optical assembly 2910 translates within shaft 2906. For example, in these embodiments, transparent section 2908 can comprise a length more than 20 mm, more than or equal to 80 mm, less than or equal to 150 mm, or less than or equal to 200 mm, such as when transparent section 2908 comprises a length of approximately 200 mm. In some embodiments, transparent section 2908 comprises a length between 60 mm and 140 mm, such as a length of approximately 80 mm or approximately 120 mm. In these embodiments in which optical assembly 2910 translates within shaft 2906, to the transparent section 2908, ID and OD of transparent section essentially match those of the opaque section 2920 (at least the portion of opaque section 2920 proximate to the transparent section), comprises an OD of less than or equal to 0.025″, 0.016″ or 0.014″. Alternatively, or additionally, in these embodiments in which optical assembly 2910 translates within shaft 2906, portions of the shaft proximal to optical assembly 2910 (e.g. proximal to transparent section 2908) can include a non-transparent construction, such as a braided construction or a construction using materials such as metal tubing (e.g. nitinol or stainless steel hypotube), such as to improve pushability of probe 200.

Console 104 can comprise an imaging assembly 128 configured to provide light to optical assembly 2910 (e.g. via rotating optical core 2900) and collect light from optical assembly 2910. Imaging assembly 128 can include a light source 126. Light source 126 can comprise one or more light sources, such as one or more light sources configured to provide one or more wavelengths of light to optical assembly 2910. Light source 126 is configured to provide light to optical assembly 212 (via rotating optical core 210) such that image data can be collected comprising cross-sectional, longitudinal and/or volumetric information related to the patient site or implanted device being imaged. Light source 126 can be configured to provide light such that the image data collected includes characteristics of tissue within the patient site being imaged, such as to quantify, qualify or otherwise provide information related to a patient disease or disorder present within the patient site being imaged. Light source 126 can be configured to deliver broadband light and have a center wavelength in the range from 800 nm to 1700 nm, from 1280 nm and 1310 nm, or approximately 1300 nm (e.g. light delivered with a sweep range from 1200 nm to 1400 nm). The light source 126 bandwidth can be selected to achieve a desired resolution, which can vary according to the needs of the intended use of system 103. In some embodiments, bandwidths are about 5% to 15% of the center wavelength, which allows resolutions of between 5 microns and 20 microns. Light source 126 can be configured to deliver light at a power level meeting ANSI Class 1 (“eye safe”) limits, though higher power levels can be employed. In some embodiments, light source 126 delivers light in the 1.3 μm band at a power level of approximately 20 mW. Tissue light scattering is reduced as the center wavelength of delivered light increases; however, water absorption increases. Light source 126 can deliver light at a wavelength approximating 1300 nm to balance these two effects. Light source 126 can be configured to deliver shorter wavelength light (e.g. approximately 800 nm light) to traverse patient sites to be imaged including large amounts of fluid. Alternatively, or additionally, light source 126 can be configured to deliver longer wavelengths of light (e.g. approximately 1700 nm light), such as to reduce a high level of scattering within a patient site to be imaged. Alternatively, or additionally, light source 126 can be configured to deliver shorter wavelengths of light (e.g. approximately 850 or 1050 nm light), such as to allow use components of retinal scanning systems.

Rotational assembly 3116 can be constructed and arranged to rotate optical core 2900 (and subsequently one or more components of optical assembly 2910) at a rotational velocity of approximately 400 rps, or at a rotational velocity between 40 rps and 1000 rps. In some embodiments, rotational assembly 3116 is constructed and arranged to rotate core 2900 at one rate (e.g. at least 100 rps or approximately 400 rps) during image data collection (i.e. an “imaging mode”), and at a different rate (e.g. a slower rate, such as a rate between 30 rps and 100 rps) during a “preview mode”. During preview mode, a “positioning operation” can be performed in which optical assembly 2910 is linearly positioned and/or a flush procedure can be initiated. The positioning operation can be configured to visualize bright reflections (e.g. via one or more implants such as an implanted stent, flow director and/or coils). Alternatively, or additionally, the preview mode can be configured to allow an operator (e.g. a clinician) to confirm that optical assembly 2910 has exited the distal end 2806 of a surrounding delivery catheter 2802. The preview mode can be configured to reduce time and acceleration forces associated with rotating core 2900 at a velocity to accommodate image data collection (e.g. a rotational velocity of at least 100 rps or approximately 400 rps).

Retraction assembly 3102 can be constructed and arranged to retract optical assembly 2910 (e.g. by retracting rotating optical core 210) at a retraction rate of approximately 50 mm s⁻¹, such as a retraction rate between 5 mm s⁻¹ and 200 mm s⁻¹. Retraction assembly 3102 can be constructed and arranged to perform a pullback of between 20 mm and 200 mm, such as a pullback that is performed in a time period between 0.5 seconds and 10.0 seconds.

Console 104 can comprise a display 132, such as a display configured to provide one or more images (e.g. video) based on the collected image data. Imaging assembly 128 can be configured to provide an image on display 132 with an updated frame rate of up to approximately 1000 frames per second (e.g. similar to the rotational velocity of rotating optical core 210). Display 132 can provide a 2-D and/or 3-D representation of 2-D and/or 3-D data.

Console 104 can comprise one or more functional elements, such as functional element shown in FIG. 1. Functional element can comprise one or more functional elements such as one or more sensors, transducers and/or other functional elements as described in detail herein.

Console 104 can comprise one or more controllers configured to read from non-transient computer-readable storage an algorithm, such as algorithm 118 shown, which can be configured to adjust (e.g. automatically and/or semi-automatically adjust) one or more operational parameters of system 103, such as an operational parameter of console 104, probe 200 and/or a delivery catheter 300. Alternatively, or additionally, algorithm 118 can be configured to adjust an operational parameter of a separate device, such as injector 106 or treatment diagnostic/delivery device 136 described herein. In some embodiments, algorithm 118 is configured to adjust an operational parameter based on one or more sensor signals, such as a sensor signal provided by a sensor-based functional element of the present disclosed concepts as described herein. Algorithm 118 can be configured to adjust an operational parameter selected from the group consisting of: a rotational parameter such as rotational velocity of rotating optical core 210 and/or optical assembly 212; a retraction parameter of optical assembly 212 such as retraction and advance velocity, distance, start position, end position and/or retraction and advance initiation timing (e.g. when retraction is initiated); a position parameter such as position of optical assembly 212; a line spacing parameter such as lines per frame; an image display parameter such as a scaling of display size to vessel diameter; a probe 200 configuration parameter; an flushing fluid 108 parameter such as a saline to contrast ratio configured to determine an appropriate index of refraction; a light source 126 parameter such as power delivered and/or frequency of light delivered; and combinations of one or more of these. In some embodiments, algorithm 118 is configured to adjust a retraction parameter such as a parameter triggering the initiation of the pullback, such as a pullback that is initiated based on a parameter selected from the group consisting of: lumen clearing; injector 106 signal; change in image data collected (e.g. a change in an image, based on the image data collected, that correlates to proper evacuation of blood from around optical assembly 212); and combinations of one or more of these. In some embodiments, algorithm 118 is configured to adjust a probe 200 configuration parameter, such as when algorithm 118 identifies (e.g. automatically identifies via an RF or other embedded ID) the attached probe 200 and adjusts a parameter such as optical path length and/or other parameter as listed above. In some embodiments, algorithm xx is configured to calculate the effect of the removal of a blockage.

Injector 106 can comprise a power injector, syringe pump, peristaltic pump or other fluid delivery device configured to inject a contrast agent, such as radiopaque contrast, and/or other fluids. In some embodiments, injector 106 is configured to deliver contrast and/or other fluid (e.g. contrast, saline and/or Dextran). In some embodiments, injector 106 delivers fluid in a flushing procedure as described herein. In some embodiments, injector 106 delivers contrast or other fluid through a delivery catheter 300 with an ID of between 5 Fr and 9 Fr, a delivery catheter 300 with an ID of between 0.53″ to 0.70″, or a delivery catheter 300 with an ID between 0.0165″ and 0.027″. In some embodiments, contrast or other fluid is delivered through a delivery catheter as small as 4 Fr (e.g. for distal injections). In some embodiments, injector 106 delivers contrast and/or other fluid through the lumen of one or more delivery catheters 300, while one or more smaller delivery catheters 300 also reside within the lumen 310. In some embodiments, injector 106 is configured to deliver two dissimilar fluids simultaneously and/or sequentially, such as a first fluid delivered from a first reservoir and comprising a first concentration of contrast, and a second fluid from a second reservoir and comprising less or no contrast. Injector 106 can comprise one or more functional elements, such as functional element shown in FIG. 1. Functional element can comprise one or more functional elements such as one or more sensors, transducers and/or other functional elements as described in detail herein.

Implant 110 can comprise an implant (e.g. a temporary or chronic implant) for treating one or more of a vascular occlusion or an aneurysm. In some embodiments, implant 110 comprises one or more implants selected from the group consisting of: a flow diverter; a Pipeline™ flow diverter; a Surpass™ flow diverter; an embolization coil; a stent; a Wingspan™ stent; a covered stent; an aneurysm treatment implant; and combinations of one or more of these. treatment diagnostic/delivery device 136 can comprise a catheter or other tool used to deliver implant 112, such as when implant 110 comprises a self-expanding or balloon expandable portion. treatment diagnostic/delivery device 136 can comprise a functional element, such as functional element 214 shown in FIG. 2. Functional element 214 can comprise one or more functional elements such as one or more sensors, transducers and/or other functional elements as described in detail herein. In some embodiments, system 103 comprises a probe 200, one or more implants 110 and/or one or more treatment diagnostic/delivery device 136 as described herein. In some embodiments, probe 200 is configured to collect data related to implant 110 and/or treatment diagnostic/delivery device 136 (e.g. implant 110 and/or treatment diagnostic/delivery device 136 anatomical location, orientation and/or other configuration data), after implant 110 and/or treatment diagnostic/delivery device 136 has been inserted into the patient.

Treatment device 114 can comprise an occlusion treatment or other treatment device selected from the group consisting of: a balloon catheter constructed and arranged to dilate a stenosis or other narrowing of a blood vessel; a drug eluting balloon; an aspiration catheter; a sonolysis device; an atherectomy device; a thrombus removal device such as a stent retriever device; a Trevo™ stentriever; a Solitaire™ stentriever; a Revive™ stentriever; an Eric™ stentriever; a Lazarus™ stentriever; a stent delivery catheter; a microbraid implant; an embolization system; a WEB™ embolization system; a Luna™ embolization system; a Medina™ embolization system; and combinations of one or more of these. In some embodiments, probe 200 is configured to collect data related to treatment device 114 (e.g. treatment device 114 location, orientation and/or other configuration data), after treatment device 114 has been inserted into the patient. Treatment device 114 can comprise a functional element, such as functional element 214 shown in FIG. 2.

Second imaging device 116 can comprise an imaging device such as one or more imaging devices selected from the group consisting of: an X-ray; a fluoroscope such as a single plane or biplane fluoroscope; a CT Scanner; an MRI; a PET Scanner; an ultrasound imager; and combinations of one or more of these.

Functional elements 214 can each comprise one or more sensors, transducers and/or other functional elements, as described in detail herein. In some embodiments, a functional element 214 is positioned proximate optical assembly 212 (e.g. distal to optical assembly 212, at the same axial location as optical assembly 212 and/or proximal to optical assembly 212). In some embodiments, imaging probe 110 comprises functional element 316 shown in FIG. 3. Functional element 316 can be positioned on a proximal portion of shaft 302 or a distal portion. Functional elements 316 can comprise one or more functional elements such as one or more sensors, transducers and/or other functional elements as described in detail herein. In some embodiments, functional element 316 comprises a sensor, such as a sensor configured to provide a signal related to a parameter of a system 103 component and/or a sensor configured to provide a signal related to a patient parameter. Functional element 316 can comprise one or more sensors selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; and combinations of one or more of these. In some embodiments, functional element 316 can comprise one or more physiologic sensors selected from the group consisting of: a pressure sensor such as a blood pressure sensor; a blood gas sensor; a flow sensor such as a blood flow sensor; a temperature sensor such as a blood or other tissue temperature sensor; and combinations of one or more of these. In some embodiments, algorithm 118 is configured to process the signal received by a sensor, such as a signal provided by a sensor as described herein. In some embodiments, the OCT signal itself is used as a pressure sensor without a separate sensor. This is calculated from the distensibility of the vessel. In some embodiments, the OCT signal itself is used as a flow sensor without a separate sensor. The flow is based on the speed blood passes the optical assembly 212. In some embodiments, functional element 316 comprises a position sensor configured to provide a signal related to a vessel path (e.g. a vessel lumen path) in three dimensions. In some embodiments, functional element 316 comprises a magnetic sensor configured to provide a signal for positioning optical assembly 212 relative to one or more implanted devices (e.g. one or more implants 110 described herein comprising a ferrous or other magnetic portion). In some embodiments, functional element 316 comprises a flow sensor, such as a flow sensor configured to provide a signal related to blood flow through a blood vessel of the patient site (e.g. blood flow through a stenosis or other partially occluded segment of a blood vessel). In these embodiments, algorithm 118 can be configured to assess blood flow (e.g. assess the significance of an occlusion), such as to provide information to a clinician regarding potential treatment of the occlusion. In some embodiments, optical assembly 212 comprises functional element 316, such as when optical assembly 212 is constructed and arranged as a sensor that provides a signal related to blood flow. In some embodiments, functional element 316 comprises a flow sensor configured to provide a signal used to co-register vessel anatomic data to flow data, which can be used to provide pre and post intervention modeling of flow (e.g. aneurysm flow), assess risk of rupture and/or otherwise assess adequacy of the intervention. In some embodiments, functional element 316 comprises an ultrasound sensor configured to provide a signal (e.g. image or frequency data) which can be co-registered with near field optical derived information provided by optical assembly 212. In some embodiments, functional element(s) 316 are configured to be deployed by their associated device, such as to implant the functional element (e.g. a sensor-based functional element) into the patient. The implantable functional element 316 can comprise microchip and/or MEMS components. The implantable functional element 316 can comprise at least a portion that is configured to be visualized (e.g. by image data collected by probe 200 and/or a separate imaging device such as second imaging device 116). In some embodiments, functional element 316 can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer to break up calcium; and combinations of one or more of these.

In some embodiments, functional element 316 comprises a pressure release valve configured to prevent excessive pressure from accumulating in the associated device. In some embodiments, functional element 316 comprises one or more sideholes xx, such as one or more sideholes xx used to deliver a fluid in a flushing procedure as described herein.

In some embodiments, functional element 316 comprises a visualizable marker, such as when functional element 316 comprise a marker selected from the group consisting of: radiopaque marker; ultrasonically reflective marker; magnetic marker; ferrous material; and combinations of one or more of these.

In some embodiments, imaging probe 100 and a second device (e.g. a diagnostic and/or treatment device), such as treatment diagnostic/delivery device 136 or treatment device 114 are positioned in a side-by-side configuration within a single delivery catheter 300, as described herein.

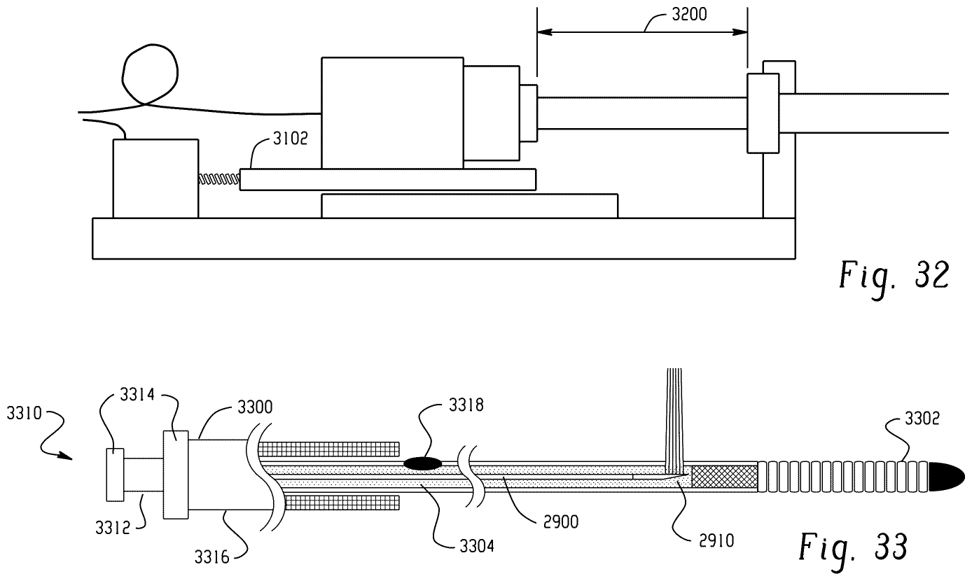

Referring now to FIG. 31 imaging probe 100 is operatively connected to rotation assembly 130 and retraction assembly 124. Rotation assembly 130 and retraction assembly 124 can comprise a linear drive assembly and a rotating motor assembly and/or other rotating mechanisms used to rotate and translate (e.g. retraction and advancement) of optical assembly 212). of core 210 of probe 200. In some embodiments, connector 208 is used to attach probe 200 to bedside unit 122 such that rotation assembly 130 can automatically attach to connector translating section 220. Operator attaches catheter by connector 208, bedside unit detects attachment and performs connecting sequence to attach to connector translating section 220 of catheter. Once connecting sequence is completed bedside unit can rotate and retract rotating optical core 210 while the shaft remains stationary. Referring now to FIG. 32 retraction assembly 3102 is shown to have pulled back rotating optical core 210 by the travel distance 3200. As shown in FIG. 29, Clamp 2902 attached to imaging probe 100 is located next to smallest delivery catheter 2904 and can be manipulated by an operator, such as to advance, retract, and/or twist shaft 2906 of probe 200, (e.g. to assist a user in advancing probe 200 to a patient site). Clamp 2902 can be loosened and/or tightened around shaft 2906, such that clamp 2902 can be repositioned along the length of shaft 2906.

As shown in FIGS. 31 and 32 rotating assembly 3116 and retraction assembly 3102 can be of similar construction and arrangement to similar assemblies used in commercial catheter-based OCT systems. One or more components of system 103 can be disposable and/or reusable. Reusable components of system 103 can be configured to be resterilizable.

### Image Core

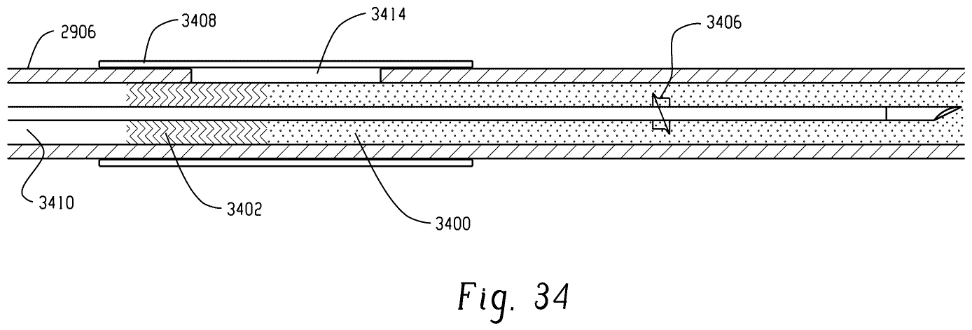

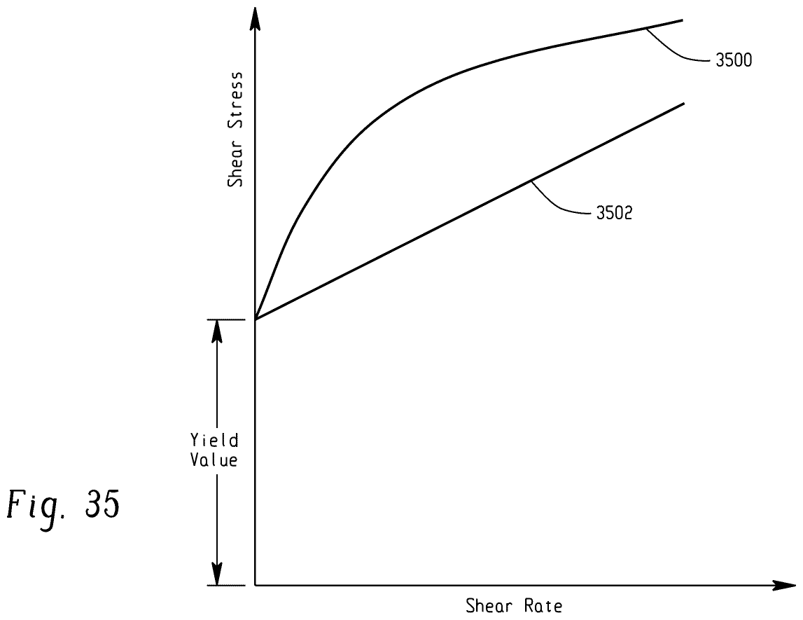

Referring now to FIG. 33, According to one aspect of the present disclosed concepts, the imaging probe comprises an elongate shaft comprising a proximal end 3300, a distal portion 3302, and a lumen 3304 extending between the proximal end and the distal portion. The imaging probe further comprises a rotatable optical core 2900 positioned within the lumen of the elongate shaft and comprising a proximal end and a distal end, the rotatable optical core 2900 configured to optically and mechanically connect with an interface unit, and an optical assembly 2910 on its distal end. The rotating optical core 2900 rotates and translates the optical assembly 2910. The proximal end 3300 of the shaft 3316 can be of a larger diameter than the distal portion 3302. Referring now to FIG. 34, in some embodiments, a liquid 3400 is included in lumen (e.g. in the space not occupied by core 2900 and optical assembly 2910), such as liquid 3400. In some embodiments, semi-solid 3402 is included. Liquid 3400 can comprise an optically transparent fluid. In some embodiments, liquid 3400 and semi-solid 3402 comprise similar materials. Alternatively, or additionally, in some embodiments, semi-solid 3402 can comprise dissimilar materials. In some embodiments, liquid 3400 comprises a less viscous fluid than semi-solid 3402. Referring now to FIG. 35 which presents an example rheologic curve of a semi solid. The x axis is the shear rate (i.e., the rate at which a material is deformed, typically measured as the velocity difference between adjacent layers divided by the distance between them). The y axis is the shear stress (i.e., the force per unit area required to cause a material to flow or deform). Yield value (approximate to yield stress) is a minimum stress a material must overcome to begin flowing if it exhibits solid-like behavior at low stresses. Line 3500 illustrates a plastic material while line 3502 exemplifies a Newtonian fluid. Axes are not to scale and are for illustrative purposes; the 0,0 origin may be regarded as the point where both lines trace from the y-axis.

Semi solid 3402 is selected from materials that have a measurable yield value before they shear. in some embodiments this is a Bingham Plastic. In some embodiments, an Ellis Plastic. The semi-solid 3402 is constructed and arranged to retain liquid 3400 during storage and/or retraction of rotating optical core 2900. Liquid 3400 can be constructed and arranged to limit undesired variations in rotational velocity of optical core 2900 and/or optical assembly 2910. In some embodiments, liquid 3400 and semi-solid 3402 are chosen to allow proximal and distal motion of the rotating optical core and optical assembly. In some embodiments liquid 3400 is selected with a viscosity of 500-3000 Cp.

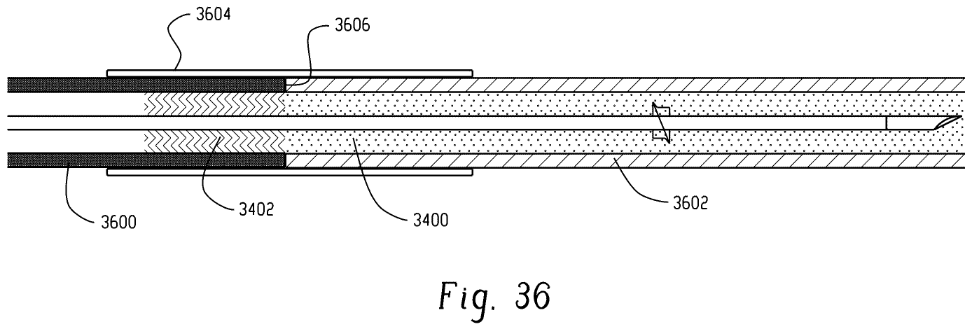

Referring now to FIGS. 34 and 36, features to introduce semi-solid 3402 are shown. FIG. 34 shows a slit 3414 cut into shaft 2906 where semi-solid 3402 is introduced during probe fabrication. Rotating optical core 2900 is placed in the probe 3310 before introduction of semi-solid 3402. Liquid 3400 may be introduced in the slit 3414 or it may be introduced from the distal end of the probe before the flexible tip 2928 is attached. The slit would act as a vent hole in that case. FIG. 36 shows an opaque sheath 3600 (nitinol) with tube 3604 joining the opaque sheath 3600 to window tube 3602 and covering/supporting joint 3606 proximate to the semi-solid 3402. Like the slit 3414, the joint allows injection of the semi-solid 3402 proximate to its final location. The joint 3606 also functions as the slit for insertion of the liquid 3400.

### Liquid Control

Pulling an optical fiber through a liquid places a large stress on the fiber. The stress is generated in two directions. The first is torsional stress. Liquid 3400 viscosity acts against the spinning of the fiber and makes the fiber twist. The second is axial stress. When the fiber is pulled back inside an imaging catheter sheath to create an image, shear is created in the liquid between the stationary shaft 2906 and the rotating optical core 2900 being pulled proximal in the sheath. The combination of these two stresses may exceed the strength of the fiber and break it.

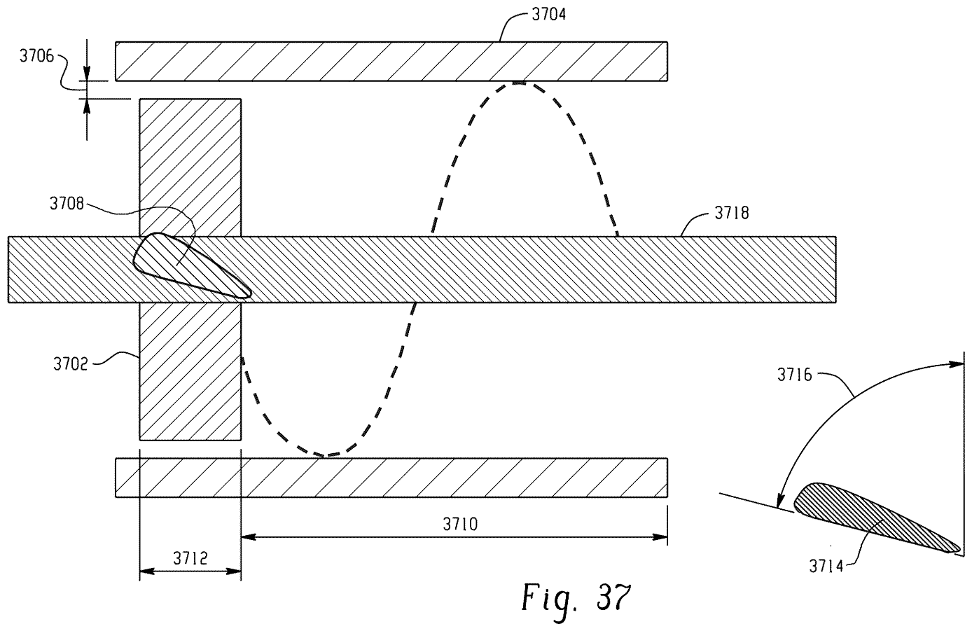

Referring now to FIG. 34, a fluid interacting element, fiber mover 3406 is attached to the rotating optical core 2900 and rotates and translates with it. Referring now to FIG. 37, the fiber mover 3702 is of a turbine design optimized to reduce tensional stress on the rotating optical core 2900 when it is being retracted during image acquisition and to aid in bringing the rotating optical core 2900 to the distal end of the catheter during readvance. The fiber mover 3702 is less than 2 mm long to allow easy passage of the matching fluid proximal with little blockage.

An example embodiment uses two blades 3708. Like a propeller in smaller boats, two blades allow the advancement of the boat with little drag. Referring to FIG. 37, the blades 3708 have a radial clearance of 0.005 to 0.002″ to probe shaft 3718. The pitch of the blades 3716 on the fiber mover is the distance the fiber mover would move in one revolution if there are no fluid losses. The actual distance the rotating optical core 2900 pulls back in one revolution is called the advance. The advance determines the spacing of the images that are obtained. The pitch of the fiber mover is selected to be slightly less than the advance to ensure the fiber mover performs effectively.

When the rotating optical core is being returned to the distal position, the direction of rotation is reversed. Then the fiber mover is then pulling the rotating optical core 2900 forward. This prevents the fiber from buckling. The fiber mover does not pressurize the liquid 3400 during imaging, it only reduces the stress on rotating optical core 2900 when it is retracted in probe shaft 3704 and helps to advance rotating optical core 2900 when it is advanced in probe shaft 3704.

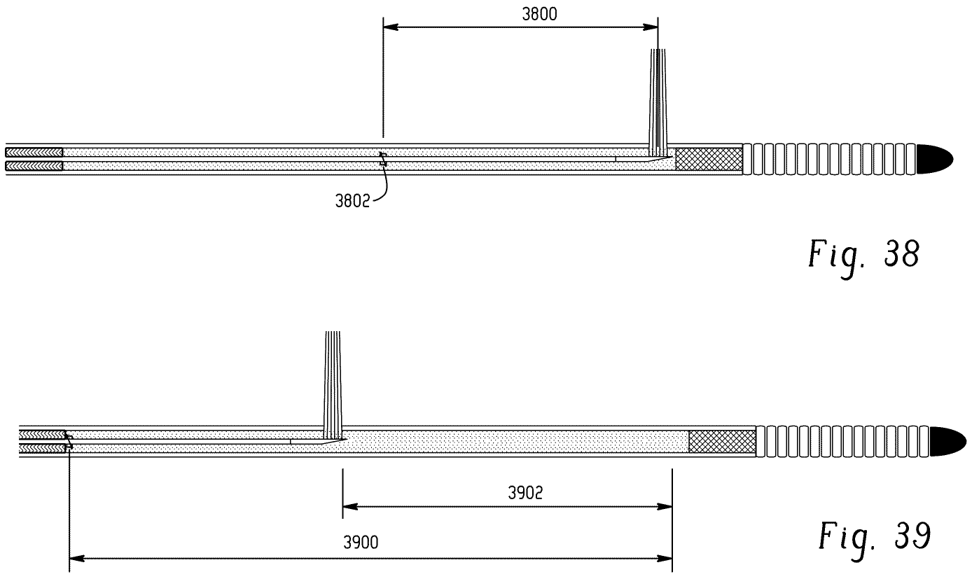

Referring now to FIGS. 38 and 39, the fiber mover 3802 is shown advanced and retracted respectively. The minimum liquid length 3900 of the low viscosity liquid is imaging distance 3000 plus the distance between the fiber mover and the tip of image core. This prevents the fiber mover 3802 from leaving the low viscosity liquid and entering the high viscosity liquid where it could get stuck. The fiber mover to optical assembly distance 3800 is selected to be great enough to prevent unwanted liquid motion around the optical assembly, but not so great as to make the minimum liquid length 3900 so great that liquid injection during construction is difficult. The fiber mover to optical assembly distance 3800 may be selected to be between about 10 mm to 50 mm.

### Optical Assembly

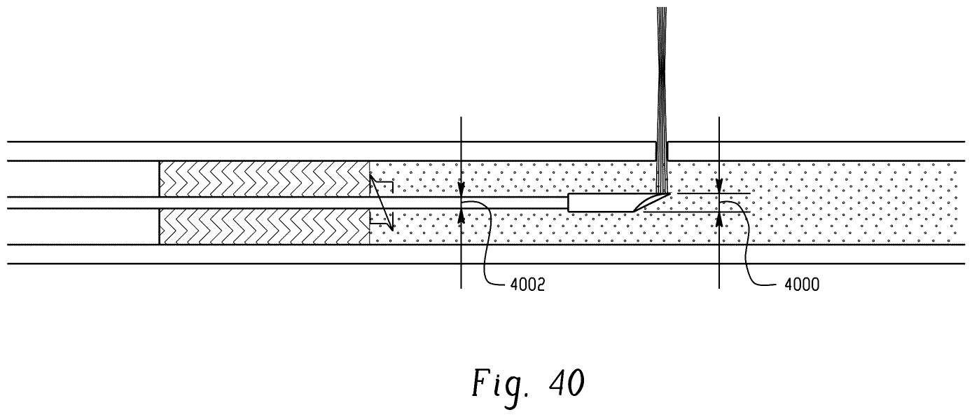

Referring now to FIG. 40, the optical assembly diameter 4000 is selected to match the rotating optical core diameter 4002 or be slightly larger. This allows axial movement of the rotating optical core 4002 without blocking liquid flow past optical assembly 2910 it, but still large enough for adequate light transmission and reception. The rotating optical core diameter 4002 is selected to be between about 60 to 80 microns and the optical assembly diameter 4000 is selected to be between about 60 to 125 microns diameter.

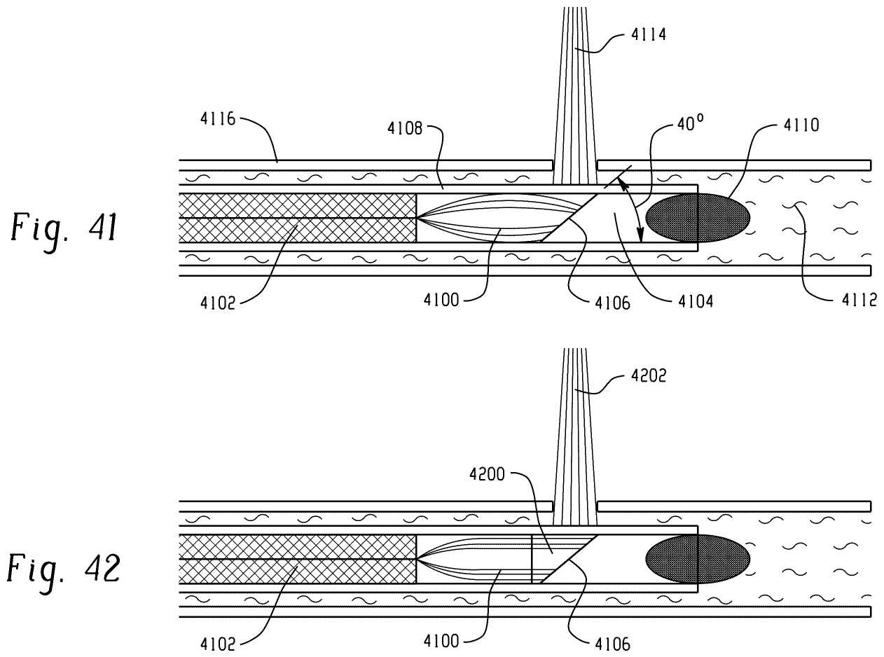

Referring now to FIG. 41, is shown an embodiment where the optical assembly diameter 4000 is selected to equal the rotating optical core diameter 4002. A graded index fiber 4100 is fusion spliced to a single mode fiber 4102. Everything distal to the single mode fiber 4102 is considered the optical assembly 2910. The distal end of graded index fiber 4100 is at an angle of roughly 40 degrees to the fiber axis. The angled surface is either coated with a reflective material or is exposed to an air gap 4104, making it reflective. Thus, it acts as an angled reflector 4106. As manufactured, single mode fiber 4102 is coated in polyimide. The polyimide is removed near proximate to splice between the graded index fiber 4100 and the single mode fiber 4102. The splice is covered by fiber protection 4108. The fiber protection 4108 goes proximal, over the polyimide. The fiber protection may be shrinkable PET (polyethylene terephthalate). It may be Pebax™. It may be nylon. The fiber protection 4108 may go distal and create a space for an air gap 4104 by the air seal 4110 on its end. The air seal 4100 keeps the air in the air gap 4104 separated from the liquid 4112.

Light 4114/4202 leaves the single mode fiber 4102, spreads out at the splice to the graded index fiber 4100 according to the fiber's index of refraction. The graded index fiber 4100 converges the light, the light reflects off the angled reflector 4106, proceeds through the fiber protection 4108, liquid, 4112, transparent section 2908 and into the vessel being imaged. At a preferential distance of 2 mm from the transparent section 2908, the light is best focused at the beam waist 4114. Light reflected from the vessel returns through the same path.

The length of the graded index fiber 4100 controls where the beam waist 4114 is located. If graded index fiber 4100 is too long the focus will be too close to the transparent section 2908. If it is too short, the focus will be too far out or may never focus at all. Controlling the length of the graded index fiber 4100 can be difficult when one end of it is an angled to create the angled reflector 4106. Referring now to FIG. 42, a coreless fiber 4200 is fusion spliced to the graded index fiber 4100 and the angled reflector 4106 is formed on it. In this embodiment the graded index fiber 4100 length may be better controlled. A “coreless” fiber is a single piece of glass with consistent optical properties throughout.

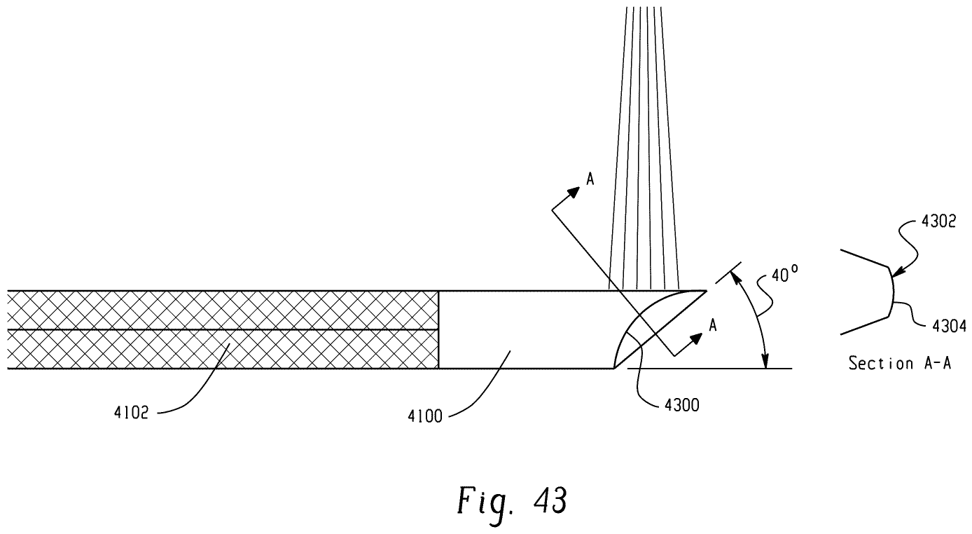

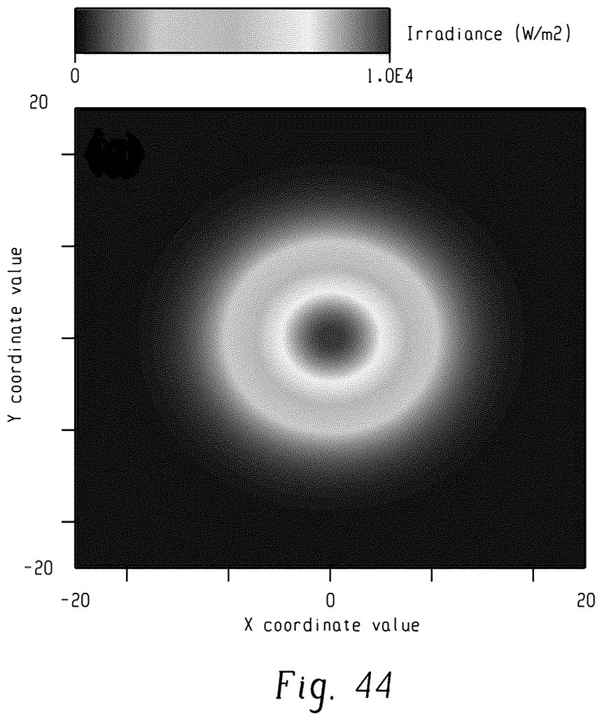

Referring now to FIG. 43, disclosed here is a curved surface 4300 formed into the graded index fiber 4100 as shown in FIG. 41, or the coreless 4200 shown in FIG. 42. This curved surface 4300 may be a single cylindrical radius 4302, made such that the 40° angle is maintained. Embodiments for the radius may be between about 0.003″ to 0.020″. The curved surface 4300 overcomes the distortion of the curved transparent section 2908 and keeps both axes focused at the beam waist. FIG. 44 is an illustration of the irradiance from the curved polish at the beam waist. The X and Y irradiance spread are equal, showing that the curved surface 4304 properly compensated for the curved transparent section 2908 distortion. At the beam waist, the 1/e² half width is 10 to 20 microns.

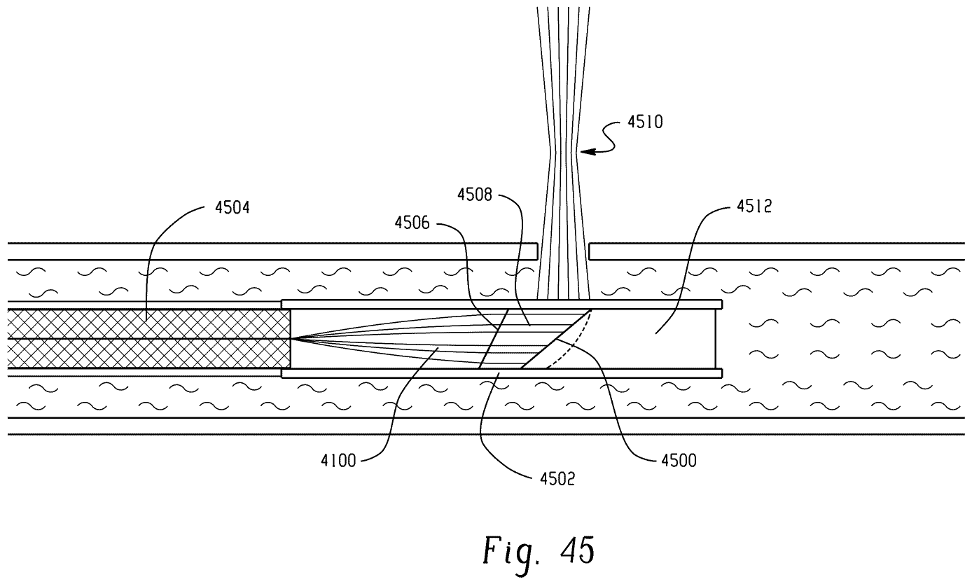

The rigid length of the lens assembly may cause difficulties advancing the optical assembly 2910, especially if the optical assembly diameter 4000 is bigger diameter than the rotating optical core diameter 4002 as shown in FIG. 40. Now referring to FIG. 45, disclosed here is a flexible lens assembly. The distal end of the graded index fiber (GRIN) 4100 is cleaved at an angle (82° to the fiber axis), the light leaves the GRIN well columnated into a fluid or flexible glue, then is turned towards the sheath by a mirror 4500. The mirror 4500 may be flat or have a concave curve to correct the astigmatism of the transparent section 2908. The mirror 4500 may be mounted inside a flexible tube 4502 to prevent a long, stiff lens assembly from binding in transparent section 2908 when the transparent section 2908 is in a sharp bend. The flexible tube 4502 may be clear PET shrunk to keep the mirror in line with the graded index fiber 4100.

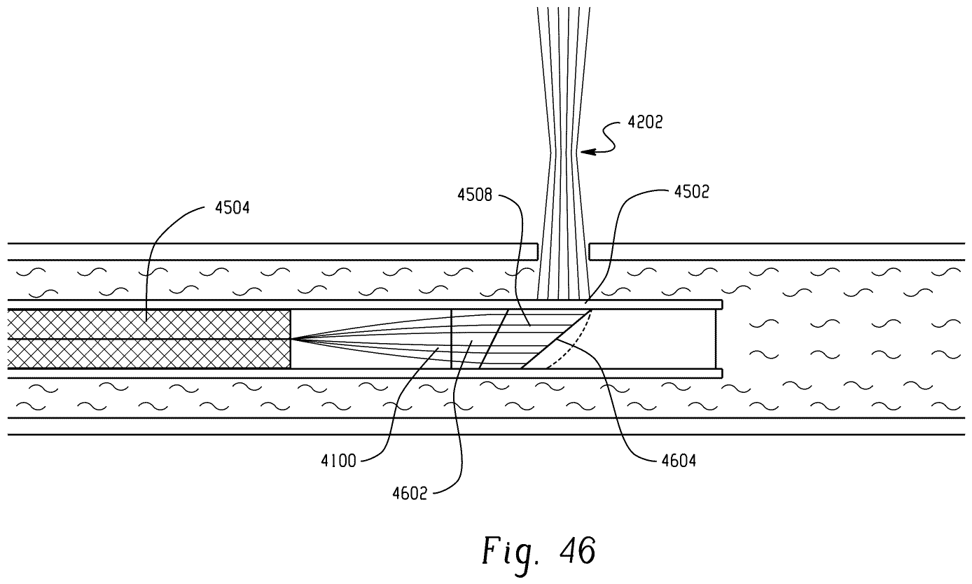

If there is a distance between the graded index fiber 4100 and the mirror 4500, the flexible tube 4502 may not be able to stay in line with the GRIN 4100. Referring now to FIG. 46, the graded index fiber 4504 is spliced to a piece of coreless 4602, the coreless 4602 length selected to bridge the gap between the GRIN and the mirror 4604. The end of the coreless is cleaved at 82° and butted against the mirror 4604. This keeps the mirror in line with the fiber. In this case, the flexible mirror mount 4502 is only able to rotate the mirror 4604 relative to the fiber and not have it translate side to side. Different embodiments of this design may include eliminating the coreless 4602 and/or the graded index fiber 4504. The 82° degree cleave angle is always placed on the last glass element, butted against the mirror 4604.

### Transparent Section

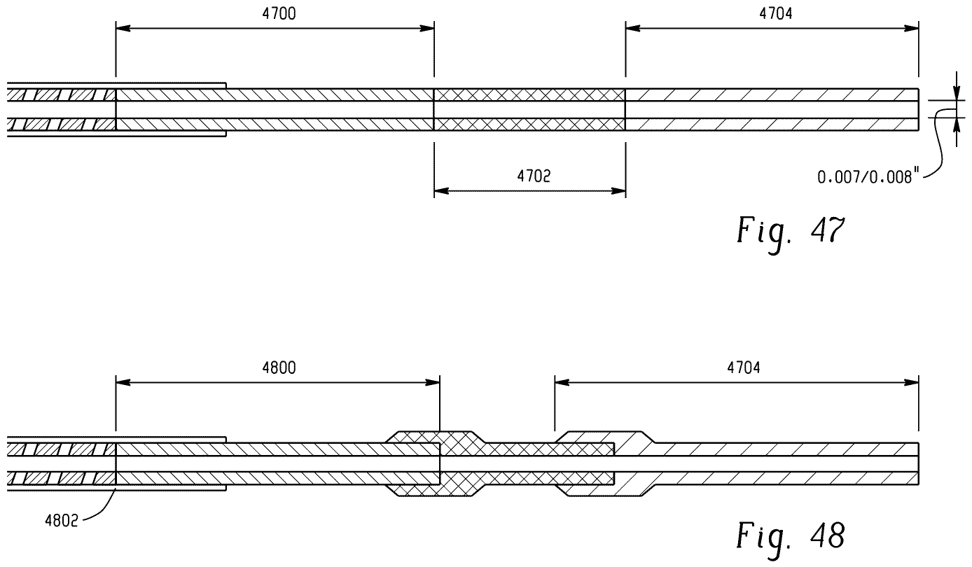

In some embodiments, transparent section 2908 of shaft 2906 can comprise a similar ID and/or OD as one or more other portions of shaft 2906. In some embodiments, transparent section 2908 comprises an inner and/or outer diameter that is smaller than other portions of shaft 2906. Transparent section 2908 comprises an OD less than or equal to 0.025″, such as an OD less than or equal to 0.022″, 0.018″, 0.016″, 0.015″ or 0.014″. It comprises a tube wall thickness less than or equal to 0.005″, such as a wall thickness less than or equal to 0.004″, 0.003″, 0.0025″, or 0.002″. Referring now to FIG. 47, in the embodiments in which transparent section 2908 comprises a relative long length, such as this present disclosure where the rotating optical core 2900 translates in shaft 2900, transparent section 2908 can comprise a material that is stiffer on the proximal end, whose stiffness matches the opaque section 2920 stiffness there. This improves deliverability of the probe 3310. Transparent section may consist of three sections, stiffest 4700, intermediate flexibility 4702, and most flexible 4704. In some embodiments, stiffest 4700 comprises a material selected from the group consisting of: amorphous polyether ether ketone (PEEK), nylon; polyethylene, polyether block amide (Pebax); and combinations of one or more of these. In some embodiments, most flexible 4704 comprises a material selected from the group consisting of nylon; polyether block amide (Pebax™), polyethylene; and combinations of one or more of these. The durometer of these materials is selected to give the desired stiffness.

Referring now to FIG. 48, the joint details between the different stiffness sections (4704, 4800) are shown. As shown, the joint may be made by flaring one of the transparent sections, inserting it over another and heat shrinking it. Alternatively, or additionally, the joint may be made by butting the tubes together and melting them. Alternatively, or additionally, the joint between transparent section 2908 and the opaque section 2920 may be made by an additional tube 4802 bridging the sections and attached to them.

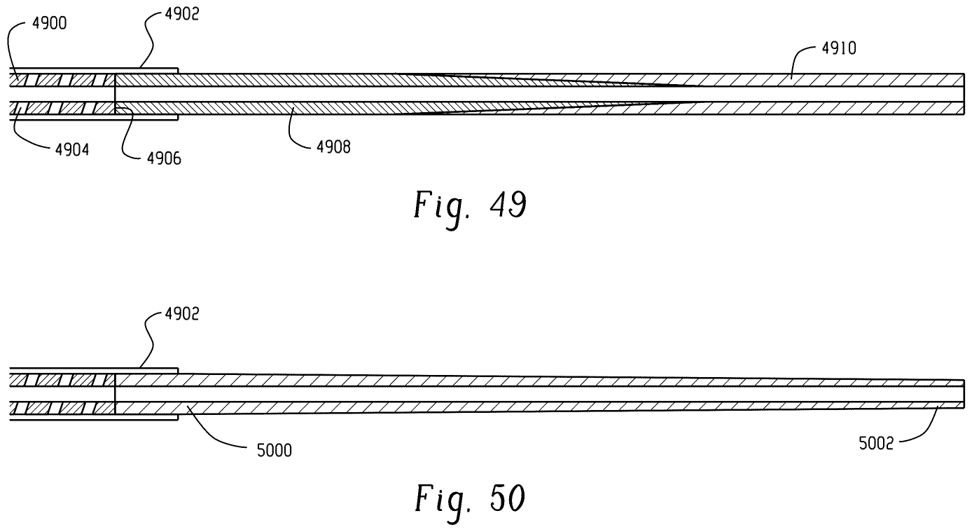

Referring now to FIG. 49, the stiffness variation is achieved by blending different materials or durometers of the same material during the tube extrusion. Materials that may be blended include in some embodiments a material selected from the group consisting of: amorphous polyether ether ketone (PEEK), nylon; polyethylene, polyether block amide (Pebax™) polyethylene; and combinations of one or more of these. In the example illustrated, joint 4906 is surrounded and supported by tube 4902 linking material 4900 with spaces 4904 to second material 4908 which transitions to third material 4910.

Referring now to FIG. 50, the stiffness variation may be achieved by varying the wall thickness of the transparent section 2908 during the tube extrusion or by a secondary operation. The tube tapers from the thickest wall 5000 proximal to the thinnest wall 5002 distal. Alternatively, the wall thickness may remain constant, and the outer diameter reduced distally. Materials that may be blended include in some embodiments a material selected from the group consisting of: amorphous polyether ether ketone (PEEK), nylon; polyethylene, polyether block amide (Pebax™) polyethylene; and combinations of one or more of these.

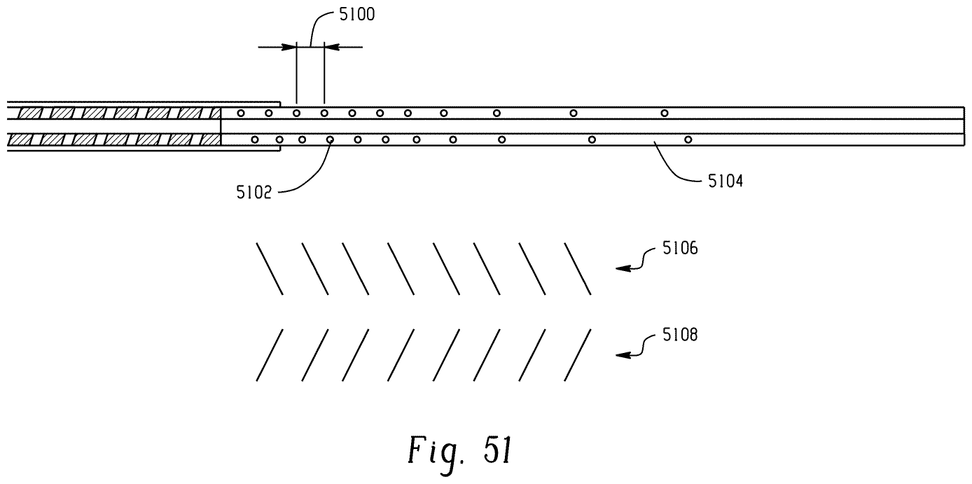

Referring now to FIG. 51, the stiffness variation may be achieved embedding a wire 5102 in the transparent section 2908. The wire 5102 may run the entire length of the transparent section or terminate in the transparent section 2908 as shown. The spiral pitch 5100 of the wire 5102 may be constant or vary with a larger pitch distal. Coil material may be a metal in some embodiments a material selected from nickel titanium alloy, stainless steel or a more radiopaque material. It also may be a plastic such as amorphous polyether ether ketone (PEEK); Torlon™; Polyimide; Vespel™; or Ultem™ some of which may be selected to be transparent to light. The wire may be embedded in window materials including in some embodiments a material selected from the group consisting of: amorphous polyether ether ketone (PEEK), nylon; polyethylene, polyether block amide (Pebax), polyethylene; and combinations of one or more of these. Should the coil be selected from a material that blocks light, it may be wound in a direction opposite the rotation direction of the rotating optical core 2900 during image acquisition. In some embodiments, the coil reinforcing assembly can be configured to resist kinking of the elongate shaft.

### Proximal Shaft

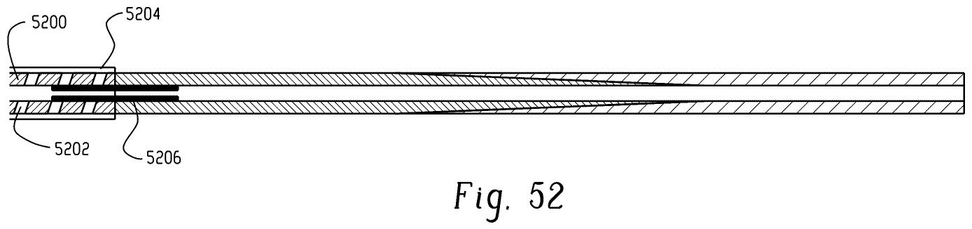

Referring now to FIG. 52, the sheath's proximal end must be a metal tube 5200 so it will have the sufficient stiffness yet still be small so as not to occlude the blood vessel being imaged. On the distal end, cuts 5202 can be made in the metal to make it more flexible and give the proper stiffness transition to improve deliverability. The cut spacing may be reduced distally to further reduce stiffness there. So that the cuts in the shaft do not present a patient risk, they are covered with a tube cover 5204. In some embodiments the cut goes around the entire circumference of the tube. In this case the tube cover 5204 is needed to have high strength as to provide tensile strength to the sheath. The metal tube can be joined to the window by a tube on its inner diameter as shown. This tube could be from the group of: polyimide, Polyamide-imide, PEEK, PET, nitinol, stainless steel or any metal. Referring to FIG. 52 the joint to the transparent section 2908 can be made by extending the tube cover 5204 over the transparent section 2908.

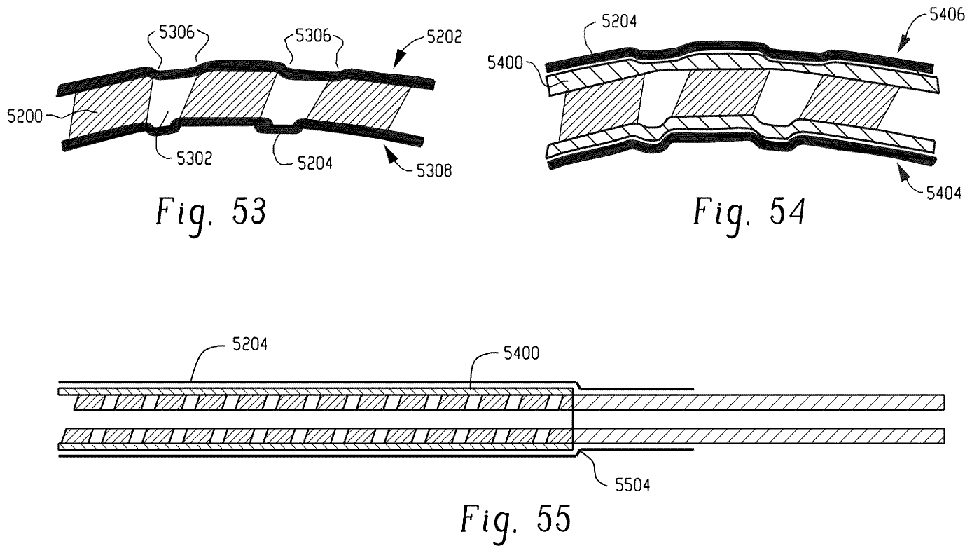

Referring now to FIG. 53, when the metal tube 5200 is bent, on the outer side 5302 of the bend, the tube cover 5304 is pulled onto the edge of the cuts 5302. The tube cover material will thin and may slide on the metal tube. This may introduce potential tears 5306 in the tube cover, which could lead to premature breakage especially if the material is notch sensitive. Referring now to FIG. 54, disclosed here is a buffer layer 5400 placed under the tube cover 5406. The buffer layer 5400 is made from a soft and stretchy material. It is thick enough to isolate the tube cover 5406 from the cuts 5302. It creates a smooth surface to protect the high strength tube cover 5406. It may be fabricated by dipping the metal tube 5300 in a polymer, shrinking a flexible polymer over the metal tube 5300 or sliding a softer polymer over the cut tube 4900. Suitable materials for these tubes are:
Buffer Layer: Pebax™, Nylon™, polyethylene, Zytel™
Tube Cover: PEEK, polyimide, PET

Referring to in FIG. 55 the transparent section joint 4802 is made only with the tube cover 5204, not the buffer layer 5400, thus the buffer tube is not required to make the joint. The outer layer of the double layer of the nitinol tube cover 5204 is further highlighted as element 5504. Alternatively, the buffer layer could be included in the transparent section joint 4802.

### Tip

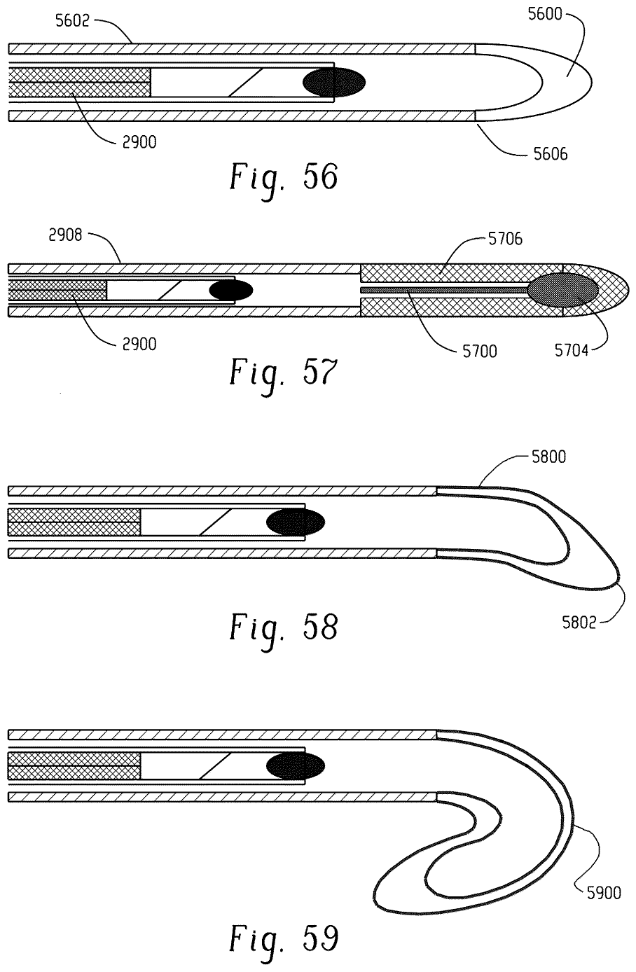

Referring now to FIG. 56, in some embodiments, a flexible tip is positioned on the distal end of transparent section 2908, such as polymer tip 5600 shown. Polymer tip 5600 can comprise a length of between 0.5 cm and 5 cm, such as a length of approximately 1 cm, 2 cm or 3 cm. At least a portion of polymer tip 5600 can be made visible to an imaging apparatus, such as by including a radiopaque material such as platinum visible to an X-ray imaging device. Polymer tip 5600 can comprise varying stiffness materials with reducing stiffness towards the tip such as different grades of Pebax™ or Nylon™. The polymer tip 5600 is attached to the transparent section 2908 without an anchor. The joint to the polymer tip is short contained in a length of between 0 mm and 2 mm, such as a length of approximately 0.5 mm, 1 mm or 2 mm. This allows the rotating optical core 2900 to be further distal than prior catheters that use an anchor to attach a spring tip.

Referring now to FIG. 57, in some embodiments the polymer tip contains a shapeable core wire 5700. The metal core may be a shapeable material such as stainless steel. Since the core wire does not take up the tensile load, it may be configured to maintain a short distance of the rotating optical core 2900 to the distal portion 3302. In some embodiments, the polymer tip 5600, is composed of the same polymer as the transparent section 2908 such Pebax™; Nylon™ and PEEK. In this case there is no joint. The polymer tip 5600 can contain materials softer than the transparent section such as lower durometer Pebax or polyurethane. Referring now to FIGS. 56, 57, 58 and 59, the polymer tip may be provided straight (FIGS. 56 and 57) or bent into a J (FIG. 58) or U (FIG. 59) shape. The J shape is so the tip may be steered. The U shape makes advancing the probe safer. The U and J straighten out when the probe is pulled proximal.

### Pressure Estimation

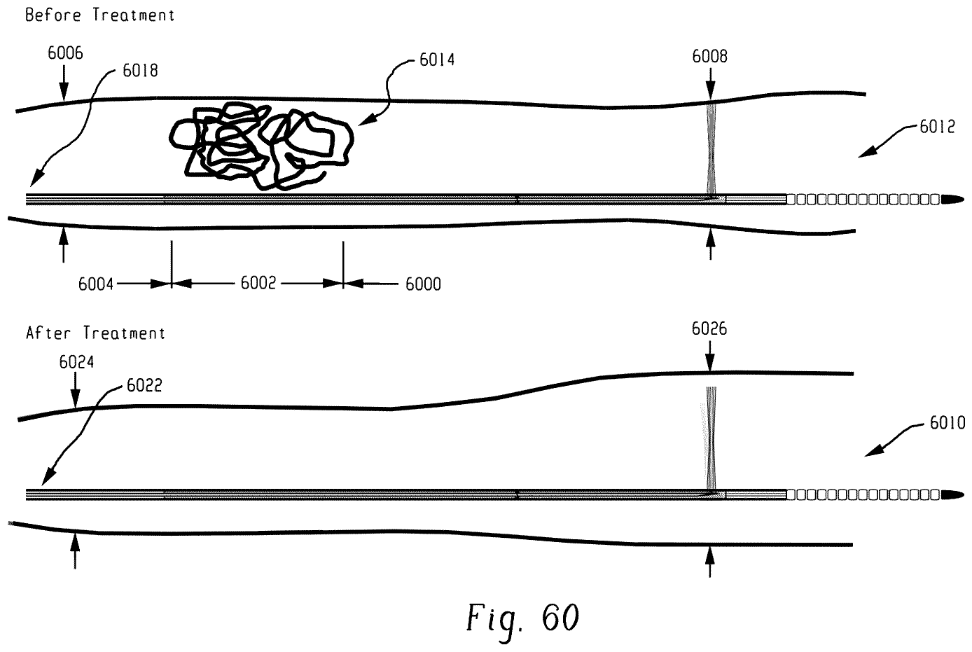

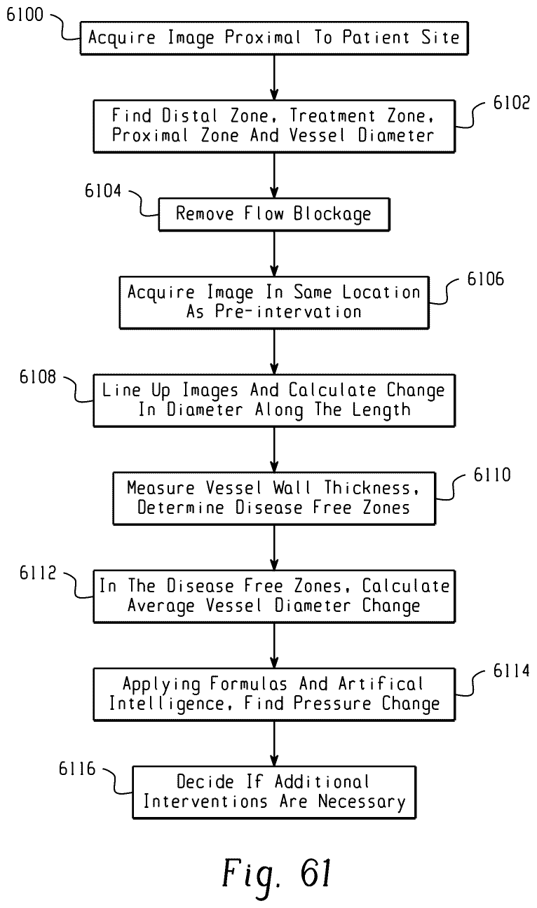

Now referring to FIG. 60, a flow blockage 6014 is shown. It is desired to know if the removal of the blockage changed the ischemia. If there is a large change, then the intervention addressed the causes of symptoms experienced by a patient. If not, then further investigation may be needed to determine the cause of the symptoms. Referring now to FIG. 61 and explained in combination with FIG. 60, shown is a flow chart of a method to determine the ischemia change. This method is applicable to images created by any system of sufficient resolution, including those made by the present disclosed concepts as described using the devices and components of system 103 described hereabove. It may be used anywhere in the body, not only intracranially. In Step 1 6100 a pre-intervention image is acquired distal area 6000 of the patient site. The image ideally includes a disease-free section of the vessel. In Step 2 6102, the image is analyzed to delineate the beginning and end of the diseased area 6002, the proximal zone 6004 and the distal zone 6000 and whether the image is sufficient for an accurate calculation. Image clarity, length of the vessel, amount of diffuse disease are potential factors that can be used to decide. This is reported to the clinician so that the clinician may determine if another image should be acquired before treatment. In Step 3 6104, the flow blockage is removed using standard intravascular techniques. In Step 4 6106, a post-intervention image is acquired in the same site as the image in Step 1 6100. In Step 5 6108, in the images from Step 1 6100 and Step 4 6106 the vessel walls are found and the vessel diameter along the length is calculated. Then the images from Step 1 6100 and Step 4 6106 are lined up. In Step 6 6110 the vessel wall thickness is measured from images previously acquired. In the images from Step 1 6100 and Step 4 6106, both the images are interrogated for disease free sections. In Step 7 6112, the average vessel diameter in the disease-free parts of the distal area 6000 is calculated in the images from Step 1 6100 and Step 4 6106. These are D-distal pre 6006 and D-distal post 6026 respectively. Likewise, the average vessel diameter in the disease-free parts of the proximal section D-proximal is calculated if available both pre and post intervention (D-proximal pre 6006 and D-proximal post 6024) respectively. In Step 8 6114, the formulas below, combined with artificial intelligence enhancements, are used to determine the pressure change from the removal of the flow blockage. In Step 9, with this information the clinician decides if the cause for the patient's symptoms have been addressed and whether additional interventions are necessary.

It is known that cerebral vessels change diameter based on blood pressure. The mean diameter change in the large cerebral arteries (carotid, middle cerebral artery, vertebral artery) is less than 4%, but the smaller arteries (anterior cerebral artery, M2 segment of middle cerebral artery) showed diameter changes as large as 21% to blood pressure changes. These arteries are especially suited to the method described here.

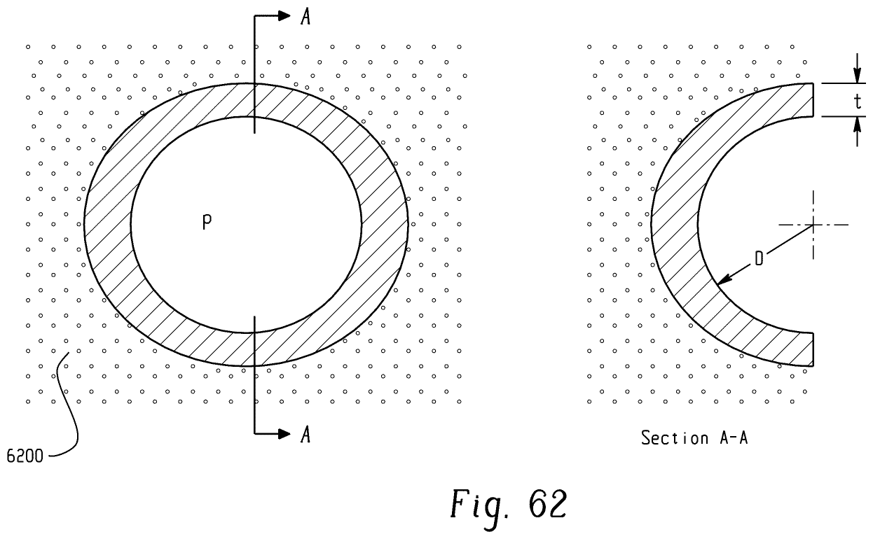

Referring now to FIG. 62, a cross section of a cerebral artery of diameter D and internal pressure P is shown. A force balance on Section A-A yields: σ=pD/2t
Where: σ=stress on the artery wall from internal pressure p
The strain ϵ on the wall is: ϵ=σ/E
Where: E is the modulus of elasticity of cerebral artery. These values are known and are based on the location of the artery in the brain, the wall thickness t, and the artery diameter D.

The diameter of the vessel when the blood pressure is zero (Do) is: D₀=D (1−ϵ)

The cerebral pressure distal to the treated area, P-distal post 6010, may be estimated from the patient's arterial pressure or simply taken as a nominal 90 mm Hg.

The cerebral pressure distal to the flow blockage prior to treatment, P-distal pre 6012, may then be calculated as:

$$P - distal pre = ( P - distal post ) \times ( {D}_{0} / D ) \times ( D - distal pre / D - distal post )$$

Defined here is a new metric, PRR, (Pressure Recovery Ratio) where:

$$PRR = P - distal pre / P - distal post$$

PRR ranges from 0 to 1. It is used as a metric to determine how much the flow blockage affected pressure. When close to 1 it implies the flow blockage was not affecting pressure in the brain and there could be other causes for the symptoms experienced by the patient.

Alternatively, or additionally PRR may be calculated by artificial intelligence applied to images distal to a flow blockage pre and post treatment where the P-distal pre is known.

### Flow Estimation

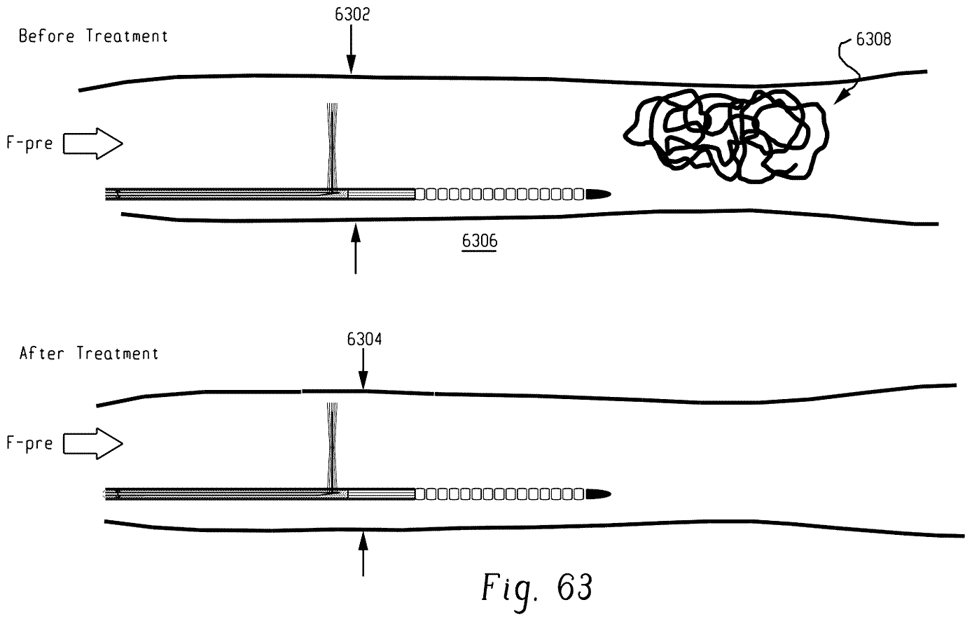

Now referring to FIG. 63, a flow blockage 6308 is shown. Again, it is desired to know if the removal of the blockage will change the amount of ischemia. This method does not necessarily require imaging, thus the image quality is of secondary importance. It uses velocity measurements made by the present disclosed concepts as described using the devices and components of system 103 described hereabove. It may be used anywhere in the body, not only intracranially. However, intracranially the flow is very steady, not pulsatile, so the methods described here are especially useful.

Velocity measurements are possible with the present disclosure through doppler techniques or simply by cross correlation of successive images of speckle without flush. The probe 200 is put in a straight section of the vessel, the rotation rate is reduced as low as possible and the correlation between successive images is used to estimate velocity. The ratio of velocity estimates taken before and after treatment at the same location is the same as the ratio of flow.

Defined here is a new metric, FRR, (Flow Recovery Ratio) where:

$$FRR = F - pre / F - post$$

FRR ranges from 0 to 1. It is used as a metric to determine how much the flow blockage affected flow. When close to 1 it implies the flow blockage was not affecting pressure in the brain and there could be other causes for the patient's symptoms.

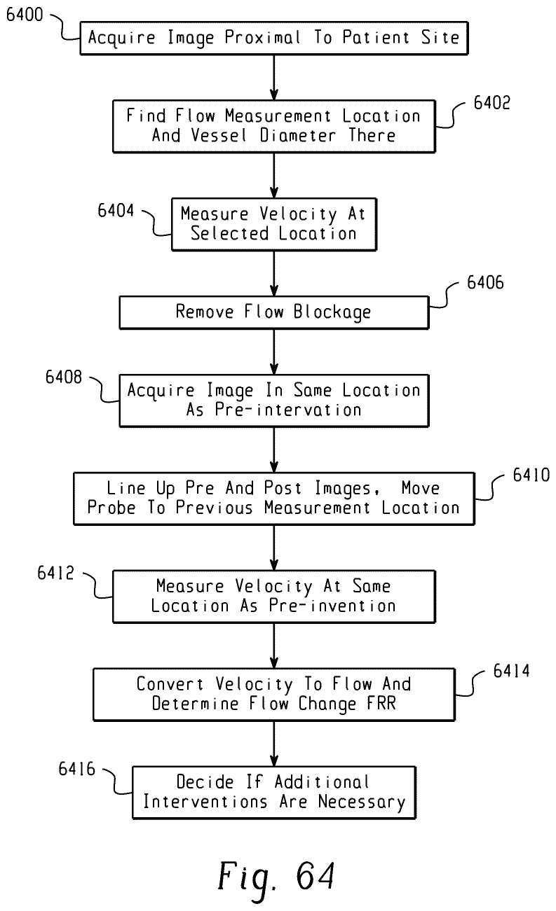

FIG. 64, shows a flow chart of a method to determine the flow change. In Step 1 6400, a pre-intervention image is acquired the proximal zone 6306 to the patient site. In Step 2 6402, the image is analyzed to find the optimum location for a velocity measurement. Ideally it is in a straight section with a relative constant diameter. sufficient for an accurate calculation. In Step 3 6404, the rotating optical core 210 is returned to optimum location. This can be done using the console and instructing it to move the optical assembly 212 to a location picked out on the display. In Step 4 6406, the flow blockage is removed using standard intravascular techniques. In Step 5 6408, a post-intervention image is acquired in the same site as the image in Step 1. In Step 6 6410 the images from Step 1 6400 and Step 5 6408 are lined up and the optical assembly 212 is returned to location where the pre-intervention image was taken. In Step 7 6412, the velocity is again measured. In Step 8 6414 the areas at the measurement locations are measured before and after intervention (A-measure pre 6302 and A-measure post 6304 respectively). These may be the same. Then the flow pre and post intervention is calculated by multiplying the measured velocities by the areas. Artificial intelligence may be used to enhance the answer's accuracy. In Step 9 6416, with this information, the clinician decides if the cause(s) for the patient's symptoms have been addressed and whether additional interventions are necessary.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein without any additional undue experimentation. It being understood that various changes may be made in the function and arrangement of elements described in an exemplary embodiment without departing from the scope of the various embodiments as set forth in the appended claims.

Since certain changes may be made in the above-described disclosure, without departing from the spirit and scope of the disclosure herein involved, it is intended that all the subject matter of the above description shown in the accompanying drawings shall be interpreted merely as examples illustrating the disclosed concept herein and shall not be construed as limiting the disclosure.

Finally, the written description uses examples to disclose the disclosure, including the best mode, and to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An imaging system comprising:
a first delivery device comprising a first elongate shaft comprising a first proximal end, a first distal end, and a first lumen extending between the first proximal end and the first distal end;
an imaging probe comprising:
a second elongate shaft comprising a second proximal end, a distal portion, and a second lumen extending between the second proximal end and the distal portion;
a rotatable optical core positioned within the second lumen of the second elongate shaft and comprising a third proximal end and a second distal end; and
an optical assembly positioned within the distal portion of the second elongate shaft and proximate the second distal end of the rotatable optical core, wherein the optical assembly is configured to direct light to tissue and collect reflected light from the tissue; and
an interface unit configured to optically and mechanically connect to the rotatable optical core, wherein the interface unit comprises:
a rotating assembly constructed and arranged to rotate the optical assembly; and
a retraction assembly constructed and arranged to retract the optical assembly separate from the second elongate shaft;
wherein the first elongate shaft and the second elongate shaft are configured to translate separately.

2. The system according to claim 1, wherein the translation of the first elongate shaft and the second elongate shaft comprises simultaneous insertion of the first elongate shaft and the second elongate shaft into a patient.

3. The system according to claim 2, further comprising a second delivery device comprising a third elongate shaft comprising a fourth proximal end, a third distal end, and a third lumen between the fourth proximal end and the third distal end, wherein the first elongate shaft and the second elongate shaft are constructed and arranged to translate within the third lumen.

4. The system according to claim 3, wherein first elongate shaft comprises a first outer diameter and the third elongate shaft comprises a first inner diameter, and wherein the first inner diameter is larger than the first outer diameter.

5. The system according to claim 1, wherein the first delivery device and the imaging probe are configured to frictionally engage.

6. The system according to claim 5, wherein the frictional engagement is configured to maintain relative position between the first elongate shaft and the second elongate shaft.

7. The system according to claim 1, wherein the first lumen of the first delivery device comprises a closed distal end.

8. The system according to claim 1, wherein the first elongate shaft further comprises a transparent segment.

9. The system according to claim 8, wherein the transparent segment comprises a length of 1 cm to 20 cm.

10. The system according to claim 1, wherein the retraction assembly retracts the optical assembly at a retraction rate of between 5 mm/sec and 150 mm/sec.

11. The system according to claim 10, wherein the retraction rate is approximately 75 mm/sec.

12. The system according to claim 1, wherein the retraction assembly performs a pullback procedure comprising retraction of the optical assembly and not the second elongate shaft.

13. The system according to claim 12, wherein the pullback procedure comprises a retraction distance of between 20 mm and 150 mm.

14. The system according to claim 12, wherein the pullback procedure is performed during a time period of between 1 second and 15 seconds.

15. The system according to claim 1, wherein one or more components of the system are at least one of a disposable component or a reusable component.

16. The system according to claim 1, wherein the one or more components comprises a reusable component that is sterilizable.

17. The system according to claim 1, further comprising a display configured to provide one or more images based on the reflected light collected by the optical assembly.

18. The system according to claim 1, further comprising:
a processor operatively coupled to the imaging probe and configured to:
(a) receive image data collected by the optical assembly from a patient site before and after a treatment procedure;
(b) determine a flow recovery ratio by comparing flow measurements derived from the image data acquired before and after the treatment procedure; and
(c) output the flow recovery ratio to a display.

19. The method of operating the system according to claim 1, comprising:
(a) acquiring first image data from a patient site using the optical assembly prior to a treatment procedure;
(b) acquiring second image data from the patient site using the optical assembly after the treatment procedure;
(c) calculating a flow recovery ratio by comparing flow measurements derived from the first and second image data; and
(d) displaying the flow recovery ratio to a user.

20. The method according to claim 19, wherein step (c) comprises calculating the flow recovery ratio (FRR) as the ratio of the flow measurement before the treatment procedure (Fₚᵣₑ) to the flow measurement after the treatment procedure (Fₚₒₛₜ), such that:
$$FRR = \frac{{F}_{p r e}}{{F}_{post}}$$
where Fₚᵣₑ and Fₚₒₛₜ are determined from velocity and cross-sectional area measurements derived from the first and second image data, respectively.
